(19) United States
(12) Patent Application Publication
(10) Pub. No.: US 2009/0238792 A1
(43) Pub. Date: Sep. 24, 2009

(54) SUPPRESSION OF POSTOPERATIVE ILEUS

(75) Inventors: Anthony J. Bauer, Pittsburgh, PA (US); Burkhard Stoffels, Pittsburgh, PA (US); Joachim Schmidt, Pittsburgh, PA (US); Savanh Chanthaphavong, Pittsburgh, PA (US); Andreas Tuerler, Bad Honnet (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: University of Pittsburgh of the Commonwealth System of Higher Education, Pittsburgh, PA (US)

(21) Appl. No.: 12/240,517

(22) Filed: Sep. 29, 2008

Related U.S. Application Data
(60) Provisional application No. 60/976,256, filed on Sep. 28, 2007.
(60) Provisional application No. 60/976,568, filed on Oct. 1, 2007.
(60) Provisional application No. 60/977,887, filed on Oct. 5, 2007.

(51) Int. Cl. A61K 38/20; A61P 41/00

(52) U.S. Cl. 424/85.2

(57) ABSTRACT

The invention provides a method for suppressing postoperative ileus in connection with the performance of a surgical procedure on a patient. In accordance with the inventive method, interleukin-10 (IL-10), glycine, a COX-2 inhibitor and a mast cell stabilizer are administered to the patient undergoing the surgical procedure in an amount and at a location sufficient to therapeutically or prophylactically suppress postoperative ileus in the patient. The invention further provides a pharmaceutically acceptable composition comprising interleukin-10 (IL-10), glycine, a COX-2 inhibitor, a mast cell stabilizer and a pharmaceutically-acceptable carrier.

### CROSS-REFERENCE TO RELATED APPLICATIONS

This application claims priority to U.S. Provisional Patent Application No. 60/976,256, filed on Sep. 28, 2007. This application also claims priority to U.S. Provisional Patent Application No. 60/976,568, filed on Oct. 1, 2007. This application also claims priority to U.S. Provisional Patent Application No. 60/977,887, filed on Oct. 5, 2007. The contents of these priority applications are incorporated herein in their entireties.

### STATEMENT REGARDING FEDERALLY SPONSORED RESEARCH AND DEVELOPMENT

This invention was made with Government support under NIH Grant Numbers R01-DK068610 (HO-1/CO), POI R01-GM58241 (POI), DK02488, and P50-GM5378 (SHOCK) awarded by the National Institutes of Health. The Government has certain rights in this invention.

### BACKGROUND OF THE INVENTION

Postoperative ileus is a transient impairment in bowel motility and is a common complication following surgery. It is a significant problem for human and veterinary medicine (particularly in equine veterinary medicine).

Symptoms of postoperative ileus include abdominal bloating, nausea, vomiting, reduced ability to pass stools, and intolerance to a solid diet. These symptoms, in turn, can result in the development of sepsis from mucosal barrier breakdown or aspiration of regurgitated gastric contents leading to pneumonia. Approximately, 15% of patients undergoing abdominal surgery digress to the ICU due to postoperative complications which can be traced to alterations in bowel function. Postoperative ileus and its associated complications lead to an increased length of hospitalization and a significant increase in the cost of postsurgical care.

Given the contribution of postoperative ileus to patient morbidity and increased expense to medical care, effective prophylaxis and treatment of postoperative ileus would be desirable. However, such prophylaxis and treatment of postoperative ileus remains elusive. Current practice currently consists primarily of supportive care by providing hydration, parenteral support and nasogastric suction of stagnant gastric contents. Accordingly, there remains a desire for a method for prophylaxis and treatment for postoperative ileus.

### BRIEF SUMMARY OF THE INVENTION

The invention provides a method for suppressing postoperative ileus in connection with the performance of a surgical procedure on a human or animal patient. In accordance with the inventive method, interleukin-10 (IL-10), glycine, a COX-2 inhibitor and a mast cell stabilizer are administered to the patient or animal undergoing the surgical procedure in an amount and at a location sufficient to therapeutically or prophylactically suppress postoperative ileus in the patient/animal. The invention further provides a pharmaceutically acceptable composition comprising IL-10, glycine, a COX-2 inhibitor and a mast cell stabilizer and a pharmaceutically-acceptable carrier.

### BRIEF DESCRIPTION OF THE SEVERAL VIEWS OF THE DRAWING(S)

FIG. 1. Fluorescence micrographs of jejunal whole-mounts immunohistochemically stained with a specific antibody against the rat glycine receptor with the secondary conjugated to Cy3. Panel A.) Muscularis whole-mounts from control rats expressed a highly fluorescent immunoreactive signal to the glycine receptor on stellate shaped cells. Panel B.) Using macrophage specific ED2 immunohistochemistry, these cells were identified as macrophages. Panel C. In addition to macrophages, enteric neuronal cells presented with glycine receptor-like immunoreactivity. Panels D and E.) Numerous glycine receptor positive infiltrating leukocytes were observed 24 hours after intestinal manipulation. Panel F.) No specific immunoreactivity was visualized using a control Cy3 staining without primary antibody incubation. (original magnification: A, C, E and F=200×; B=250×; D=100×).

FIG. 2. Gastrointestinal transit histograms and calculated geometric center histogram for the distribution of fluorescein-labeled dextran along the gastrointestinal tract 2 hours after oral administration (SM: intestinal manipulation, St=stomach, SB=small bowel, CM=cecum, C=colon). Panel A.) Gastrointestinal transit distribution histograms for control and glycine pretreated control showing the progression of the transit marker to the distal regions of the small intestine over a period of 2 hours. Panel B.) Gastrointestinal transit distribution histograms for vehicle and glycine pretreated animals which had been surgically manipulated 24 hours prior to measurement. Vehicle pretreatment resulted in a marked delay in gastrointestinal transit. Glycine pretreatment significantly improved postoperative gastrointestinal transit with the fluorescent marker transported more caudally compared to the vehicle pretreated manipulated rodents (N=6 each). Panel C.) Histogram demonstrating calculated transit geometric centers (GC) for five groups of rats. Control and glycine pretreated non-operated rats had a geometric center approximately 10 indicating transit near to the cecum. Surgical manipulation (SM) with vehicle treatment significantly reduced the GC, while this reduction was prevented by glycine immunomodulation pretreatment. In contrast, valine did not significantly improve gastrointestinal transit. (N=6 each, mean±SEM; F-test and Bonferroni post-hoc group comparisons with *=p-values<0.05).

FIG. 3. Representative bethanechol stimulated (100 μM) jejunal circular smooth muscle contractile activity recorded in an organ bath 24 hours after surgical manipulation (SM). A vigorous contractile response is observed in vehicle injected rats controls (Panel A). Intestinal manipulation in vehicle animals caused a distinct reduction of the contractile response to bethanechol (Panel B), whereas manipulated rats after glycine pretreatment (Panel C) presented with only a mild reduction in bethanechol stimulated contractile activity. Valine pretreated animals also exhibited a distinct reduction of the contractile response to bethanechol (Panel D).

FIG. 4. Bethanechol dose response curves of jejunal circular smooth muscle contractile activity 24 hours after surgical manipulation (SM) with glycine or valine pretreatment. Panel A shows a robust control response curve (∘) calculated from control jejunal muscles. Surgical manipulation with saline pretreatment significantly decreased muscle contractility (). Glycine pretreatment at both 85 mg/kg (▾) and 170 mg/kg (▪) dose dependently prevented the suppression in muscle contractility. Panel B illustrates that valine pretreatment did not markedly change in vitro muscle contractility. (n=5-11 per group, data are expressed as mean±SEM).

FIG. 5. Histograms of real-time two-step RT-PCR analysis of inflammatory mediator mRNA expressions 3 hours postoperatively (SM: intestinal manipulation). The mRNA of the three inflammatory indicators were qualified (IL-6, TNF-α and ICAM-1) in unoperated controls, after surgical manipulation and glycine pretreatment of the surgically manipulated animals. Each inflammatory mRNA was markedly induced by surgical manipulation and this induction was significantly blunted by glycine pretreatment. (n=6-8 per group, data are expressed as mean±SEM).

FIG. 6. FIG. 6A illustrates myeloperoxidase (MPO) staining of jejunal muscularis whole-mounts demonstrating few cells in the control (A) and significant leukocyte recruitment after intestinal manipulation (B) and a diminution in neutrophil recruitment with glycine pretreatment of surgically manipulated animals (C). (A, 100×; B-C, 40×). FIG. 6B shows a histogram of quantified infiltrating MPO-positive leukocytes in muscularis whole mounts from vehicle or glycine injected control rats versus surgically manipulated rats pretreated with vehicle/saline, glycine (85 mg/kg and 170 mg/kg) or valine (263.5 mg/kg) pretreatment (SM=surgical manipulation). Glycine pretreatment significantly attenuates postoperative leukocyte recruitment into the intestinal muscularis (n=6-8 per group, data are expressed as mean±SEM; F-test and Bonferroni post-hoc group comparisons with *=p-value<0.05).

FIG. 7. Histograms of real-time two-step RT-PCR analysis of inflammatory mediator mRNA expressions for iNOS and COX-2 3 hours postoperatively (SM: surgical manipulation). The mRNAs of both synthases were significantly induced by SM. Glycine radically suppressed the postsurgical induction of iNOS (p<0.05) and tended to suppress COX-2 at this single time point. (n=6 per group, data are expressed as mean±SEM; F-test and Bonferroni post-hoc group comparisons with *=p-value<0.05).

FIG. 8. Histograms showing the Gries reaction measurement of nitrite and ELSIA for prostanoids release into the 24 hour organ culture media by the intestinal muscularis with and without intestinal manipulation and glycine or valine. Panel A reflects an increased release of nitric oxide from the organ cultured surgically manipulated muscularis externa over control unoperated tissue of the jejunum and colon. Glycine pretreatment substantially suppressed the postoperative increase in nitric oxide production in both the jejunum and colon. Valine has no effect on the production of nitrite from the tissue. Panel B show a similar result of significant glycine suppression of prostanoid production measure by ELISA for both the jejunal and colonic muscularis externa in organ culture. Valine tended to decrease prostaglandin release by this was not significant. (n=6 per group, data are expressed as mean±SEM; F-test and Bonferroni post-hoc group comparisons with *=p-value<0.05).

FIG. 9. Histograms depicting glycine quelling of interleukins (Panel A) and chemokines (Panel B) released into the organ culture media of manipulated jejunal muscularis externa. All interleukins and chemokines tended to be quelled with statistical significance reached by IL-6, IP-10 and MIP1-α.

FIG. 10. Histograms depicting glycine quelling of interleukins (Panel A) and chemokines (Panel B) released into the organ culture media of manipulated colonic muscularis externa. All interleukins and chemokines tended to be quelled with statistical significance reached by IL-5, IL-6, IL-10, IL-12, KC, MCP-1, MIP1-α and GM-CSF. (n=5 per group, data are expressed as mean±SEM; F-test and Bonferroni post-hoc group comparisons with *=p-value<0.05).

FIG. 11. Early augmentation of IL-1β mRNA within the surgically manipulated intestinal muscularis externa. Real time 2-step RT-PCR analysis for IL-1β mRNA expression in the isolated muscularis externa from un-operated IL-10+/+ and IL-10−/− mice and from the muscularis obtained 3 hours after surgical anesthesia and surgical manipulation of IL-10+/+ and IL-10−/− mice. The relative fold increase calculated from the ΔCT values show a significant induction of IL-1β mRNA in wild-type IL 10+/+mice (*) and a significant augmentation of this induction in IL-10−/− mice (†). Data are expressed as mean±SEM with ANOVA, Bonferroni post-hoc comparison (*=p<0.01 relative to control; †=p<0.01 relative to control and IL-10+/+SM, N=4 each).

FIG. 12. Gastrointestinal transit distribution histograms from unoperated wild-type C57BL/6 (WT) mice and IL-10−/− (KO) mice without and with surgical manipulation of the small intestine after 24 hours (POD-1). Mice from both genetic backgrounds had a similar and significant delay in gastrointestinal transit (N=5 each, see FIG. 4 for statistics).

FIG. 13. Gastrointestinal transit distribution histograms from unoperated wild-type C57BL/6 (WT) mice and IL-10−/− (KO) mice without and with surgical manipulation of the small intestine on postoperative day 5 (POD-5). In wild-type mice, gastrointestinal motility recovered by POD 5 from surgical manipulation. In contrast, IL-10−/− mice continued to display a significant delay in gastrointestinal transit (N=5 each, see FIG. 4 for statistics).

FIG. 14. Mean calculated geometric centers from individual gastrointestinal transit distribution histograms from 8 groups of animals (unoperated wild-type C57BL/6 (WT) mice and IL-10−/− (KO) mice without and with surgical manipulation of the small intestine on postoperative days 1, 3 and 5). Wild-type animals displayed an initial and significant decrease in the calculated geometric center reflecting the suppression in gastrointestinal transit on POD-1, with recovery from ileus evident on POD-3 and resolution on POD-5. IL-10−/− mice displayed an initial and significant decrease in the calculated geometric center reflecting the suppression in gastrointestinal transit on POD-1 that was not different from wild-type mice. Recovery from ileus in the IL-10−/− mice appeared to proceed more slowly on POD3 and on POD-5 a significant difference in the resolution from ileus was statistically calculated compared to wild-type on POD-5 (ANOVA, Bonferroni post-hoc comparison, *=p<0.05 compared to wild-type and IL 10−/− mice, †=p-value<0.05, comparing wild-type and IL-10−/− on POD-5, N=6 each).

FIG. 15. Mechanical activity of small intestinal circular smooth muscle strips recorded in vitro in response to bethanechol (100 μM) from unoperated wild-type C57BL/6 (WT) mice and IL 10/− (KO) mice without and with surgical manipulation of the small intestine on postoperative day 5. Panels A and B: Representative mechanical traces from control wild-type and IL-10−/− mice showing robust bethanechol stimulated contractile activity. Panel C: Typical mechanical trace from a control wild-type mouse on postoperative day 5 after intestinal manipulation showing resolution of ileus by generating a robust contractile response to bethanechol. Panel D: Typical mechanical trace from an IL-10−/− mouse on postoperative day 5 after intestinal manipulation showing a direct and persistent suppression in the contractile response to bethanechol.

FIG. 16. Bethanechol stimulated dose-response curves of mechanical activity recorded in vitro from small intestinal circular smooth muscle strips of unoperated wild-type C57BL/6 (WT) mice and IL 10/− (KO) mice without and with surgical manipulation of the small intestine on postoperative days 1 and 5. Dose-response curve generated in postoperative day 1 (▪) muscles exhibited the typical previously observed suppression in jejunal muscle contractility. On postoperative day 5 after surgical manipulation (▴), IL-10−/− muscles continued to exhibit a significant suppression in jejunal muscle contractility.

FIG. 17. The mRNAs for pro-inflammatory (IL-6, IL-1β and CCL2) and anti-inflammatory (HO-1) mediators were quantified by SYBR green real-time two-step RT-PCR analysis in the isolated muscularis externa of unoperated wild-type C57BL/6 (WT) mice and IL 10/− (KO) mice without and with surgical manipulation of the small intestine on postoperative days 1, 3 and 5. Panels A, B and C show significantly augmented inductions of IL-6, IL-1β and CCL2 mRNAs after surgical manipulation in IL-10−/− mice at various time points through postoperative day 5 after surgery compared to wild-type mice. Panel D shows the enhanced mRNA induction of the anti-inflammatory mediator HO-1 on postoperative day 1 after surgical manipulation in IL-10−/− mice compared to wild-type mice. Data are expressed as mean±SEM (ANOVA, Bonferroni post-hoc comparison, *=p<0.05 comparing wild-type and IL 10−/− mice, N=6 each).

FIG. 18. A murine Twenty-Plex Luminex assay was used to quantify the release of inflammatory proteins (FGF basic, GM-CSF, IFN-γ, IL-1α, IL-1β, IL-2, IL-4, IL-5, IL-6, IL-10, IL-12p40/p70, IL-13, IL-17, IP-10, KC, MCP-1, MIG, MIP-1α and TNF-α) into the media of the 24 hour organ cultured muscularis externa harvested from unoperated wild-type C57BL/6 (WT) mice and IL 10−/− (KO) mice without and with surgical manipulation of the small intestine on postoperative days 1, 3 and 5. The Luminex measurement of 6 pro-inflammatory cytokines are illustrated in Panels A-F (IL-6, IL-1α, TNF-α, IL-12, IL-17, IFN-γ) and 3 chemokines (CCL2, IP-10 and GM-CSF) in Panels G-I showing their significant elevations following surgery and their augmented production in IL-10−/− mice compared to wild-types at various time points through postoperative day 5 after surgery (ANOVA with Bonferroni post-hoc comparison *=p<0.05, N=5 each).

FIG. 19. Enhanced release of nitric oxide and prostanoids into the media of the 24 hour organ cultured muscularis externa harvested from unoperated wild-type C57BL/6 (WT) mice and IL 10/− (KO) mice without and with surgical manipulation of the small intestine on postoperative days 1, 3 and 5. The histogram bars in Panel A show the significant increase in nitric oxide levels in the culture media assayed by the Griess reaction from the postsurgical muscularis harvested on postoperative days 1 and 3. In IL-10−/− mice, the amount of nitric oxide released was significantly greater on postoperative days 1 and 3 compared to wild-type animals. The histogram bars in Panel B show the significant increase in prostanoid levels in the culture media assayed by ELISA from the postsurgical muscularis harvested on postoperative days 1, 3 and 5. In IL-10−/− mice, the amount of prostaglandins released by the postsurgical inflamed muscularis was significantly greater on postoperative days 1, 3 and 5 compared to wild-type animals (ANOVA with Bonferroni post-hoc comparison *=p-value<0.05, N=6 each).

FIG. 20. Quantification of infiltrating neutrophils into full thickness whole-mounts of the inflamed postsurgical muscularis externa by Hanker-Yates myeloperoxidase histochemical staining dissected from unoperated wild-type C57BL/6 (WT) mice and IL 10/− (KO) mice without and with surgical manipulation of the small intestine on postoperative days 1, 3 and 5. Wild-type mice displayed a significant recruitment of neutrophils into the muscularis externa which peaked on postoperative day 3 showed significant signs of resolution on postoperative day 5. Whole-mounts from IL-10−/− mice demonstrated a postsurgical infiltration of neutrophils which was quantitatively less than wild-types and which did not show any resolution (ANOVA with Bonferroni post-hoc comparison *=p-value<0.05, N=5 each).

### DETAILED DESCRIPTION OF THE INVENTION

In accordance with the inventive method, the patient can be a human or animal (i.e., veterinary or research model), for example a cat, dog, horse, pig, goat, cattle, or other animal.

The inventive method can be used adjunctively with any surgical procedure associated with post-operative ileus, such as cardiac surgery, brain surgery, cancer surgery, gastrointestinal surgery, orthopedic surgery, gynecological surgery or other surgical procedures. Such procedures are within the ken of those of ordinary skill in the art. The type of surgical procedure, thus, is not critical for practice of the inventive method.

Regardless of the type of surgical procedure, in accordance with the inventive method, the patient is treated by administration of IL-10, glycine, one or more COX-2 inhibitor(s), and one or more mast cell stabilizer(s).

Without wishing to be bound by theory, it is believed that postoperative ileus results from the surgical initiation of a complex innate inflammatory response within the muscularis externa. Constitutively present mast cells and the dense network of resident muscularis macrophages which normally lie quiescent within the mesentery and gut muscle layer throughout the gastrointestinal tract are activated by a surgical procedure, and this results in the activation of inflammatory transcription factors with a subsequent induction of chemokines (such as CCL), cytokines (such as IL-6, IL-1β, TNF-α), release of smooth muscle kinetically active mediators (nitric oxide and prostaglandins generated by iNOS and COX-2, respectively) and mast cell mediators. This locally generated inflammatory milieu upregulates vascular adhesion molecules resulting the recruitment and extravasation of circulating leukocytes which further participate in the suppression of gastrointestinal motility. The inventive method consists of suppressing the innate inflammatory state of the resident dense network of macrophages and mast cells within the abdominal cavity, which causes and perpetuates the clinical state of postoperative ileus.

In the context of the present invention, a COX-2 inhibitor can be any agent that inhibits the COX-2 enzyme, such as any nonsteroidal anti-inflammatory agent (NSAIDs—Diclofenac, Etodolac, Indometacin, Nabumetone, Sulindac, and the like) or more selective agents. Preferably, however, a COX-2 inhibitor for use in the context of the present invention is at least partially selective for COX-2, as opposed to COX-1. Exemplary COX-2 inhibitors include, but are not limited to celecoxib, meloxicam, rofecoxib, and valdecoxib.

Human muscularis externa, unlike in rodents, has a large constitutive presence of “resident” mast cells. It is believed that both resident populations play a role in human postoperative ileus. Therefore, the use of a mast cell stabilizer is included as a therapeutic substance for human patients. For use in the context of the present invention, a mast cell stabilizer can be any agent that prevents the release of mediators from mast cells. Exemplary mast cell stabilizers include, but are not limited to azelastine, cromolyn, nedocromil, ketotifen, lodoxamide, nedocromil, olopatadine, and pemirolast. Of course, pharmaceutically-acceptable salts, hydrates, polymorphs, and the like of the COX-2 inhibitors and mast cell stabilizers can be used in the context of the present invention (for example, azelastine hydrochloride, cromolyn sodium, nedocromil sodium, ketotifen fumarate, lodoxamide tromethamine, nedocromil sodium, olopatadine hydrochloride, and pemirolast potassium are pharmaceutically acceptable forms of various mast cell stabilizers).

Pharmaceutical grade preparations of these reagents, and formulations are commercially available. And any of them can be used in the context of the inventive method. Suppliers of such reagents, for example, are as indicated in the following table:

TABLE 1

| | Raw API | Suitable |
|---|---|---|
| Ingredient | supplier/tradename | formulation/tradename |
| IL-10 | Ilodecakin/Tenovil | Schering Plough |
| Glycine | Glycine | Baxter/SigmaAldrich |
| COX-2 inhibitors | | |
| Celecoxib | Celebrex | Pfizer |
| Meloxicam | Mobic | Boehringer Ingelheim |
| Rofecoxib | Vioxx | Merck |
| Valdecoxib | B extra | Pfizer |
| Mast cell stabilizers | | |
| azelastine | Astelin | medPointe Pharmaceuticals |
| hydrochloride | | |
| cromolyn sodium | Nasalcrom | Pfizer |
| nedocromil sodium | Alocril; Tilade | Allergan |
| ketotifen fumarate | Zaditor | Novartis |
| lodoxamide | Alomide | Alcon |
| tromethamine | | |
| pemirolast potassium | Alamast | Vistakon Pharmaceuticals |

In performing the inventive method, IL-10, glycine, COX-2 inhibitor(s) and mast cell stabilizer(s) are administered to the patient in an amount and at a location sufficient to suppress postoperative ileus in the patient. These agents can be delivered, for example, orally (e.g., as a tablet or capsule), parenterally, or topically (e.g., directly to the bowel), as desired. Topical delivery to the bowel is preferred, however, to minimize systemic effects (i.e. allow healing of the bowel anastomosis and laparotomy).

In accordance with the inventive method, one or more of IL-10, glycine, COX-2 inhibitor(s) and mast cell stabilizer(s) can be delivered to the patient after the onset of postoperative ileus, to effect a therapeutic suppression of the condition. However, the inventive method can also be used prophylactically by administering one or more of the IL-10, glycine, COX-2 inhibitor(s) and mast cell stabilizer(s) prior to the surgical procedure or onset of the symptoms of postoperative ileus. For example, one or more of the IL-10, glycine, COX-2 inhibitor(s) and mast cell stabilizer(s) can be administered to the patient following the surgical procedure, such as prior to completion of healing of the surgical wound (e.g., within a few days of the surgical procedure). More typically, one or more of the IL-10, glycine, COX-2 inhibitor(s) and mast cell stabilizer(s) are administered contemporaneously with the surgery, such as during the surgery (e.g., after laparotomy, prior to the closure of the surgical incision(s). Or IL-10, glycine, COX-2 inhibitor(s) and mast cell stabilizer(s) can be administered prior to the surgical incision (e.g., within a few hours of the incision). Additionally, one or more of the IL-10, glycine, COX-2 inhibitor(s) and mast cell stabilizer(s) may be administered at multiple pre/post-surgical time points.

Of course, one or more of the IL-10, glycine, COX-2 inhibitor(s), mast cell stabilizer(s), and (optionally) biliverdin can be administered at different times relative to the surgery, and they need not be administered at the same time. IL-10, glycine, COX-2 inhibitor(s) and mast cell stabilizer(s) can be delivered to the patient together or separately, and some of the agents can be co-delivered while others are delivered separately. However, it is preferred for the IL-10, glycine, COX-2 inhibitor(s) and mast cell stabilizer(s) to be administered to the patient substantially concurrently (such that there is overlap in their administration). It is more preferably for the IL-10, glycine, COX-2 inhibitor(s) and mast cell stabilizer(s) to be administered to the patient in a single pharmaceutical formulation.

The IL-10, glycine, COX-2 inhibitor(s) and mast cell stabilizer(s) can be administered to the patient at any suitable dosage, which can readily be determined by one of ordinary skill in the art. Moreover, the desired dosage will depend on the mode and route of administration. However, exemplary and non-limiting dosages of the various agents are presented in the following table:

TABLE 2

| Ingredient | Route of Administration | Dosage range | |
|---|---|---|---|
| IL-10 | i.v. or i.p or topical | 30-500 | μg/L |
| Glycine | i.v. or i.p or topical | 0.5-5.0 | g/L |
| COX-2 inhibitors | | | |
| Celecoxib | i.v. or i.p or topical | 30-250 | mg/L |
| Meloxicam | i.v. or i.p or topical | 30-250 | mg/L |
| Rofecoxib | i.v. or i.p or topical | 30-250 | mg/L |
| Valdecoxib | i.v. or i.p or topical | 30-250 | mg/L |
| Mast cell stabilizers | | | |
| azelastine | i.v. or i.p or topical | 0.05-0.5% | solution |
| hydrochloride | | | |
| cromolyn sodium | i.v. or i.p or topical | 1-7.5% | solution |
| nedocromil sodium | i.v. or i.p or topical | 0.5-5% | solution |
| ketotifen fumarate | i.v. or i.p or topical | 0.5-5% | solution |
| lodoxamide | i.v. or i.p or topical | 0.05-0.25% | solution |
| tromethamine | | | |
| pemirolast potassium | i.v. or i.p or topical | 0.05-0.5% | solution |

As noted, the inventive method results in therapeutic or prophylactic suppression of postoperative ileus in the patient. By “suppression,” it is not required for the inventive method to eliminate postoperative ileus, however. It is sufficient, and a therapeutically acceptable outcome, for the inventive method to lessen the severity or duration of the ileus or any of its symptoms. Similarly, it is prophylactically acceptable for the inventive method to lessen the likelihood that a patient will develop postoperative ileus and/or to lessen the severity or duration of the ileus or any of its symptoms should the ileus develop despite prophylactic use of the inventive method.

For use in the inventive method, IL-10, glycine, COX-2 inhibitor(s) and mast cell stabilizer(s) should be administered to a patient as one or more pharmaceutically acceptable formulations. Such formulations of these agents separately are already known, and such existing formulations can be employed. Additionally, it is within the competence of a person or ordinary skill in the art to formulate the agents, separately or together, for administration to a patient via a desired route. Accordingly, the invention further provides a pharmaceutical composition comprising, as active agents, IL-10, glycine, COX-2 inhibitor(s), mast cell stabilizer(s) and a pharmaceutically-acceptable carrier.

Ideally, the active agents, IL-10, glycine, COX-2 inhibitor(s), mast cell stabilizer(s) and a pharmaceutically-acceptable carrier would be carried in an encapsulated time release form administered via a clinging foam which rapidly melts into the tissues upon which it is sprayed. The clinging spay foam would localize the application of the “active agents” (here meaning—IL-10, glycine, COX-2 inhibitor(s), mast cell stabilizer(s)) avoiding a systemic effect (i.e. substantially sparing immunological interference as the site of bowel anastomosis and incision). Avoiding a systemic effect of the “active agents” would allow the bowel and incision site to heal and prevent a systemic suppression in immunological function. The encapsulated time release formulation would ideally deliver the “active agents” over the duration of the surgical procedure and for a period of a few hours after incisional closure. The active release formulation would in a sustained manner locally deliver the “active agents” to the tissue generating a local concentration of IL-10 (5-20 mg/liter), glycine (150-300 mg/liter), COX-2 inhibitor(s) (1-15 mg/liter), mast cell stabilizer(s) (0.1-15 mg/liter) with a non-immunogenic pharmaceutically-acceptable carrier.

The pharmaceutically acceptable compositions of this invention can be administered to humans and other animals orally, rectally, parenterally, intraperitoneally, laproscopically or topically (as by spray, gel, foam, powders, ointments, or drops), or the like, depending on the skill in the art of appropriate practice and treatment. The inventors expect skilled artisans to employ such variations as appropriate, and the inventors intend for the invention to be practiced otherwise than as specifically described herein.

Components of the anti-inflammatory agents may individually be given in different forms and administered via different routes. Dosage forms for oral, subcutaneous, intramuscular, intravenous or intraperitoneal administration include, but are not limited to, pharmaceutically acceptable foams, emulsions, microemulsions, solutions, suspensions, syrups, powders and elixirs. In addition to the active compounds, the dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, corn, germ, olive, castor, and sesame oils), glycerol, tetrahydrofurfuryl alcohol, polyethylene glycols and fatty acid esters of sorbitan, and mixtures thereof. Besides inert diluents, the compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable or foam depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly (orthoesters) and poly (anhydrides). Depot injectable and foam formulations are also prepared by entrapping the compound in liposomes or microemulsions that are compatible with body tissues.

Compositions for rectal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical administration of a compound of this invention include ointments, pastes, creams, lotions, gels, powders, solutions or sprays. The active component is admixed under sterile conditions with a pharmaceutically acceptable carrier and any needed preservatives or buffers as may be required. Absorption enhancers can also be used to increase the flux of the compound across the intestinal serosa. The rate can be controlled by either providing a rate controlling membrane or by dispersing the compound in a polymer matrix or gel.

The following examples further illustrate the invention but, of course, should not be construed as in any way limiting its scope.

### Example 1

This example demonstrates a direct bowel application of all administered agents immediately after laparotomy for a bowel resection procedure.

The patient is anesthetized and a laparotomy is performed. Immediately there after, a adherent foam containing the agents (IL-10 500 μg/l, glycine 2 g/l, COX-2 inhibitor (Celebrex 250 mg/l), mast cell stabilizer (ketotifen fumarate 0.1%) in a 1% ethanol and optionally choline chloride solution) would be sprayed/coated directly onto the bowel wall avoiding the region in which the anatomosis will take place, hence to allow normal immune reparative mechanisms to not be altered at the anastomotic site of healing. In this formulation, ethanol is added as a solvent for the COX-2 inhibitor and because it would increase the open probability of the glycine gated chloride channel. The optional choline chloride solution may, in some embodiments, be used to increase the driving force of chloride through the glycine gated chloride channel on the immune cells (e.g., macrophages, monocytes, neutrophils, etc). When used, the concentration of the choline chloride solution preferably is less than about 7.5%. As immunosuppression with glycine, COX-2 inhibition and mast cell stabilization) will down-regulate pro-inflammatory mediators of ileus, additionally, the agents will decrease endogenous anti-inflammatory mediators (IL-10). It is, therefore, preferable that IL-10 be given in conjunction with the immunosuppressants. The surgical procedure would then continue as the art demands. At the end of the procedure, the anti-ileus foam would again be supplied to the bowel wall, again avoiding the anastomotic site. Then, the laparotomy would be closed to end the surgical procedure. The above example is a preferable scenario, as normal reparative mechanisms at both the site of anastomosis and laparotomy would be allowed to proceed uninhibited by the local bowel wall application of the anti-ileus foam.

### Example 2

This example demonstrates the therapeutic potential of exogenous IL-10 to prevent POI.

Methods: Mice were subjected to surgical intestinal manipulation (SM) to induce ileus. Before and after animals were treated with IL-10 (12.5 μg/kg, s.c.). Gastrointestinal transit and organ bath measured motility. Histochemistry on jejunal muscularis whole-mounts quantified neutrophil recruitment. Muscularis mediator expressions were measured by RT-PCR, Griess reaction, ELISA and Luminex (N=4 each).

Results: SM caused a delay in transit, exogenous IL-10 treatment prevented the delay in transit (GC: control=10.6±0.3 vs. SM=4.6±0.6 vs. SM+IL 10=10.8±0.4). SM resulted in a suppression of jejunal circular muscle contractions to bethanechol (59.2±4.8% of control at 100 μM), which was improved by IL-10 treatment (93.6±21.1%). Muscularis neutrophil recruitment was significantly less with IL-10 treatment compared to SM with vehicle. A significant upregulation in IL-6, IL-1β and MCP 1 mRNAs after SM was detected by SM compared to controls with less induction in IL-1β treated animals. SM induced a significant increase in mediator release (GM-CSF, VEGF, IL-1α, IL-6, MCP-1 and MIP-1α), which were significantly diminished by IL-10 treatment. NO and prostanoids were diminished by IL-10 (control-NO=47.1; SM-NO=607.7; SM+IL-10=NO: 49.1 μM/g and control-PG=4033.4; SM-PG=13111; SM+IL-10-PG=3946.7 pg/ml/mg).

Conclusion: Exogenous IL-10 prevents the development of rodent postoperative ileus by reducing molecular and cellular innate immune events. Pre-emptive exogenous IL-10 could be potential therapeutic treatment for the prevention of clinical postoperative ileus.

### Example 3

This example demonstrates that preoperative glycine reduces postoperative ileus.

### Methods

Animals and Operative Procedures: Male ACI (black agouti) rats (180-220 g) and C57Bl/6 mice (20-30 g) were obtained from Harlan (Indianapolis, Ind.). The experimental design was approved by the University of Pittsburgh Institutional Animal Care and Use Committee (IACUC). All animals were kept in a pathogen-free facility that is accredited by the American Association for Accrediation of Laboratory Animal Care and complies with the requirements of humane animal care as stipulated by the U.S. Department of Agriculture and the Department of Health and Human Services. They were maintained on a 12-hour light/dark cycle and provided with standard laboratory rodent chow and tap water ad libitum.

Glycine, dissolved in normal saline (170 mg/kg) was injected intravenously via the penile vein 1 hour prior to surgery. This dose has shown to increase glycine serum levels about sevenfold and to be effective in the prevention of reperfusion injury in rat liver transplantation 28. Some additional animals were treated with a lower dose of glycine (85 mg/kg). Age matched animals treated with vehicle (normal saline) or valine (263.5 mg/kg, osmotic control) at appropriate time points and at equal volumes served as controls.

The rodent intestine was subjected to a standardized degree of surgical manipulation as described previously (14). Rats were anesthetized by isoflurane inhalation and the abdomen was opened by a midline laparotomy. The cecum was eventrated and laid out onto a moist gauze. Then, the intestine was inspected along its length and gently compressed using moist cotton applicators. The midline incision was closed by two layers of continuous sutures. All operative procedures were performed under sterile conditions with the intestine being handled only by instruments and never touched directly. Animals were killed at specific time points postoperatively by isoflurane anesthesia inhalation overdose and heart incision.

Bowel Preparation Rodents were anesthetized by isoflurane inhalation and the abdomen was opened by a midline laparotomy. The jejunum and colon were removed and placed in iced preoxygenated Krebs-Ringer buffer (KRB). Functional studies described below were carried out immediately on bowel specimens taken from the mid-jejunum. For gene expression and protein analyses, the intestinal muscularis was isolated from the mucosa-submucosa by slipping 5-cm-length portions of the intestine over a glass rod and stripping the muscularis from the jejunal mucosa, as described previously (22). The isolated muscularis was snap-frozen in liquid nitrogen and stored at −80° C. or organ cultured for Luminex analysis. Three jejunal segments per animal were used for histochemical and immunohistochemical analysis.

Functional Studies Rat and mouse in vivo gastrointestinal transit was measured at 24 hours postoperatively by evaluating the gastric and intestinal distribution of orally administered fluorescein-labeled dextran as previously described, (N=4-9) 18. The data were expressed as the percentage of activity per segment and plotted in a histogram. Gastrointestinal transit was calculated as the geometric center (GC) of distribution of fluorescein-labeled dextran using the following formula:

GC=Σ(% of total fluorescent signal per segment*segment number)/100

The GC value reflects the transit of fluorescently-labeled dextran down the gastrointestinal tract, with higher values indicating more distal distribution (segment 1=stomach to segment 15=distal colon).

Mechanical in vitro activity of the rat mid-jejunum was evaluated at 24 hours postoperatively using smooth muscle strips of the circular muscularis as described previously (rats: N=5-11) (14). After dissection and a 30 minute equilibration period, the individual muscles were incrementally stretched to L0. Then after recording spontaneous contractility for 30 minutes, dose response curves were generated using increasing concentrations of the muscarinic agonist bethanechol (1-300 mmol/L) for 10 minutes and intervening wash periods (KRB) of 10 minutes. The contractile response was recorded and analyzed as g/mm2/sec over a 10 minute period.

mRNA Expression Analysis: Postoperative mediator mRNA expressions in the small intestinal muscularis were analyzed using SYBR Green two-step real-time RT-PCR as previously described, (N=4-6). Based on our previous observation that postoperative mediator induction reaches a maximum between 2 and 6 hours postoperatively, comparative mRNA analysis was performed at 3 hours. Total mRNA extraction was performed using the guanidium-thiocyanate phenol-chloroform extraction method. Equal aliquots (200 ng) of total RNA from each sample, quantified by spectrophotometer, were processed for complimentary DNA (cDNA) synthesis. Primer sequences were used as described previously (tumor necrosis factor-a, TNF-a) (35), or designed according to published sequences 36-41, (GenBank accession numbers: NM_{—}017008 [glyceraldehyde phosphate dehydrogenase (GAPDH)], AF233596 [cyclo-oxygenase-2 (COX-2)], NM_{—}012967 [intercellular adhesion molecule-1 (ICAM-1)], M26744 [interleukin-6 (IL-6)] and NM_{—}012611 [inducible nitric oxide synthase (iNOS)]), using Primer Express software (PE Applied Biosystems, Foster City, Calif.) and purchased from Life Technologies (Rockville, Md.). GAPDH was used as an endogenous control. The sequences of the real-time-PCR primers are listed in Table 3. The efficiency and equality of the real-time PCR primer pairs were determined by amplifying serial dilutions of muscularis cDNA. For each target gene different MgCl2 (3 to 5 mM) concentrations were tested to optimize the PCR amplification. Agarose gel electrophoretic analysis was used to verify the presence of a single product and that the amplified product corresponded to size predicted for the amplicon. The PCR reaction mixture was prepared using the SYBR Green PCR Core Reagents (PE Applied Biosystems, Foster City, Calif.). PCR conditions on an ABI PRISM 7700 Sequence Detection System (PE Applied Biosystems, Foster City, Calif.) were as recommended by the manufacturer. Each sample was estimated in duplicate. Dissociation of the PCR products by a melting curve analysis protocol consistently showed specific single melting peaks for all primer pairs. Relative quantification was performed using the comparative CT method as described by Schmittgen et al. (42).

TABLE 3

Nucleotide sequences of oligonucleotide primers.
Slopes and correlation coefficients of the
primer standard curves.

| Target | | | | Correlation- |
|---|---|---|---|---|
| gene | Primer sequences (5′ to 3′) | MgCl₂ | Slope | coefficient |
| GAPDH | Sense: ATGGCACAGTCAAGGCTGAGA (SEQ | 3 | -3.197 | 0.996 |
| | ID NO: 1) | | | |
| | Antisense: CGCTCCTGGAAGATGGTGAT (SEQ | | | |
| | ID NO: 2) | | | |
| COX2 | Sense: CTCTGCGATGCTCTTCCGAG (SEQ ID | 5 | -3.494 | 0.991 |
| | NO: 3) | | | |
| | Antisense: AAGGATTTGCTGCATGGCTG (SEQ | | | |
| | ID NO: 4) | | | |
| ICAM-1 | Sense: CGTGGCGTCCATTTACACCT (SEQ ID | 3 | -3.404 | 0.991 |
| | NO: 5) | | | |
| | Antisense: TTAGGGCCTCCTCCTGAGC (SEQ ID | | | |
| | NO: 6) | | | |
| IL-6 | Sense: GCCCTTCAGGAACAGCTATGA (SEQ ID | 5 | 3.360 | 0.997 |
| | NO: 7) | | | |
| | Antisense: TGTCAACAACATCAGTCCCAAGA | | | |
| | (SEQ ID NO: 8) | | | |
| INOS | Sense: GGAGAGATTTTTCACGACACCC (SEQ | 3 | -3.518 | 0.999 |
| | ID NO: 9) | | | |
| | Antisense: CCATGCATAATTTGGACTTGCA | | | |
| | (SEQ ID NO: 10) | | | |
| TNF-α | Sense: GGTGATCGGTCCCAACAAGGA (SEQ ID | 5 | -3.021 | 0.997 |
| | NO: 11) | | | |
| | Antisense: CACGCTGGCTCAGCCACTC (SEQ ID | | | |
| | NO: 12) | | | |

Histochemistry and Immunohistochemistry: Leukocyte infiltration was quantified on rat whole-mounts of the intestinal muscularis as described previously (N=6-8) (43). Myeloperoxidase (MPO) positive cells were detected using a mixture of 30 mg Hanker-Yates reagent, 30 ml KRB and 300 ml 3% hydrogen peroxide for 10 minutes. Leukocytes were counted in 5 randomly chosen areas in each specimen (3 specimen per animal) at a magnification of 200×.

For glycine-receptor-immunohistochemistry rat jejunal whole mounts were incubated for 18 hours at 4° C. with a mouse-anti-rat monoclonal glycine receptor antibody (1:50), followed by 3× washes in 0.05 mol/l phosphate-buffered saline solution (PBS). Each specimen was then incubated with a Cy3 donkey-anti-mouse secondary antibody (1:500) for 4 hours at 4° C. and washed again three times in 0.05M PBS. Secondary antibodies without glycine receptor antibody preincubation were used in parallel in all staining procedures to ensure specificity. Macrophages were visualized by using ED2 immunohistochemistry, as previously described (19).

Tissue culture and determination of nitrite and prostanoid release in supernatant: The rat small intestine and colon were processed for tissue culture at 24 hours as described previously, (N=5-8) (25). Briefly, the muscularis was isolated by stripping the muscularis from the mucosa and then cut into small pieces and washed twice in Hank's balanced salt solution (HBSS) with 100 U/ml penicillin G and 100 μg/ml streptomycin. Aliquots of muscularis (80-120 g) were incubated (37° C., 5% CO2) in 6-well tissue culture plates containing 3 ml of supplemented Dulbecco's modified Eagle medium (DMEM)-F12 with 100 U/ml penicillin G and 100 μg/ml streptomycin. After an incubation period of 24 hours, 500 μl aliquots of supernatant were collected, frozen in liquid nitrogen and stored at −80° C. The muscle tissue was blotted dry, and the weight was estimated. To estimate the generation of NO from the intestinal muscularis, nitrite production was measured by the Griess reaction (44). Supernatant from the tissue culture was mixed with equal amount of Griess reagent (0.1% N-(1-naphtyl)ethyl-enediamine dihydrochloride and 1% sulfanilamide in 6% phosphoric acid; 1:1) and incubated at room temperature for 10 minutes. The absorbance at 550 nm was measured with a microplate reader and compared with standard prepared from serial dilutions of sodium nitrite. Each standard and supernatant sample was analyzed in duplicate, and the mean value of nitrite production was normalized to the tissue weight.

The synthesis of prostanoids released into the tissue culture supernatant over a period of 24 hours was assayed by ELISA. After incubation the culture median and tissue was inspected, 500 μL aliquots of supernatant were frozen in liquid nitrogen and stored at −80° C. The muscle tissue was blotted dry and weighed. Culture supernatants were assayed for the measurement of prostaglandin secretion by enzyme immunoassay (Cayman Chemicals, Ann Arbor, Mich.). The ELISA was carried out in a 1:10 and 1:30 dilution. The assay was corrected for wet tissue weights and the prostaglandin ELISA kit sensitivity was 20 pg/ml.

Luminex Multiplex Bead Immunoassays: The release of 22 inflammatory analytes into the mouse tissue culture supernatant, obtained as above, was quantified with a Luminex 100™ using microsphere-based multiplexing technology. The mouse cytokine twenty plex immune kit was comprised of analyte specific components for the simultaneous measurement of the following mouse cytokines (FGF-basic, IL-1α, IL-1β, IL-2, IL-4, IL-5, IL-6, IL-10, IL-12p70, IL-13, IL-17, TNF-α, IFN-γ, GM-CSF, KC, MCP-1, MIG, MIP-1α, IP-10 and VEGF) (Invitrogen, Carlsbad, Calif.).

Drugs and solutions: A standard KRB was used as described previously 18,43. KRB constituents, bethanechol, glycine, L-valine, urea, 3% hydrogen peroxide, HBSS and DMEM-F12 were purchased from Sigma Chemical Co. (St. Louis, Mo.). Fluorescein-labeled dextran (molecular weight=70,000) was purchased from Molecular Probes (Eugene, Oreg.). Hanker-Yates reagent was obtained from (Polysciences, Warrington, Pa.). The mouse-anti-rat monoclonal antibody to glycine receptor (all subunits) was purchased from Alexis Corp. (San Diego, Calif.) and the mouse-anti-rat ED2 antibody was purchased from Serotec Inc. (Raleigh, N.C.). Indocarbocyanine (Cy3)-conjugated donkey-anti-mouse antibody was obtained from Jackson ImmunoResearch Laboratories Inc. (West Grove, Pa.). For immunohistochemistry antibodies were diluted in 0.05 mol/L PBS containing 0.2% bovine serum albumin (Sigma Chemical Co., St. Louis, Mo.), 1000 U/ml penicillin G, and 1 mg/ml streptomycin (Sigma Chemical Co., St. Louis, Mo.).

Data analysis: Results are expressed as mean±standard error of the mean (SEM). The data were analyzed using student-t test or analysis of variance (ANOVA). Statistical analysis was performed using a Student t-test (paired for the COX-2 and L-NIL inhibition experiments and unpaired for all other experiments). EZAnalyze add-in for Microsoft Excel was used for F-test and Bonferroni post-hoc group comparisons where appropriate. p values<0.05 were considered significant.

### Results

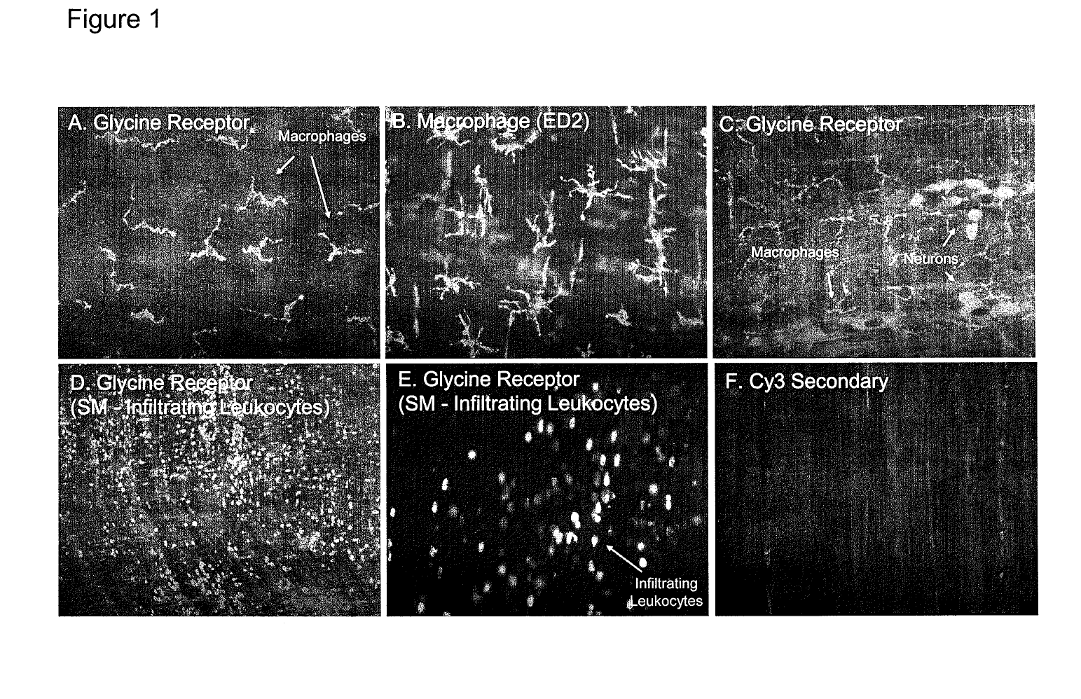

Glycine receptor in the intestinal muscularis: To clarify if cellular receptors for glycine exist within the intestinal muscularis which could be a potential target for the immunosuppressive effects of glycine 45, we first investigated the localization of glycine receptors within the intestinal muscularis using immunohistochemistry. As shown in FIG. 1A, muscularis whole-mounts from control animals exhibited glycine receptor-like immunoreactivity prominently on stellate shaped cells. The distribution and morphology of these cells were similar to that of resident muscularis macrophages and immunohistochemical staining with the rat macrophage marker ED2 confirmed the identification of these cells as resident muscularis macrophages (FIG. 1B). Glycine receptor immunoreactivity was also recognized in a subpopulation of neurons in the myenteric plexus (FIG. 1C). Under normal physiological circumstances infiltrating leukocytes are nearly absent in the intestinal muscularis. However, after a local or systemic injury to the gut, such as surgical manipulation (46), endotoxemia (47,48), ischemia/reperfusion injury (49) or hemorrhagic shock (50), a significant number of leukocytes are recruited into the intestinal muscularis. Thus, we also stained for glycine receptor immunoreactivity 24 hours after surgical manipulation of the intestine to determine the presence of the glycine receptor on infiltrating leukocytes. As depicted in FIGS. 1D and 1E, whole-mounts after intestinal manipulation showed pronounced receptor-like immunoreactivity on round to oblong shaped cells with a distribution and morphology resembled those of infiltrating leukocytes. Staining procedures with the secondary antibody but without glycine receptor antibody preincubation revealed no specific immunoreactivity either on macrophages or neurons in control whole-mounts (FIG. 1F).

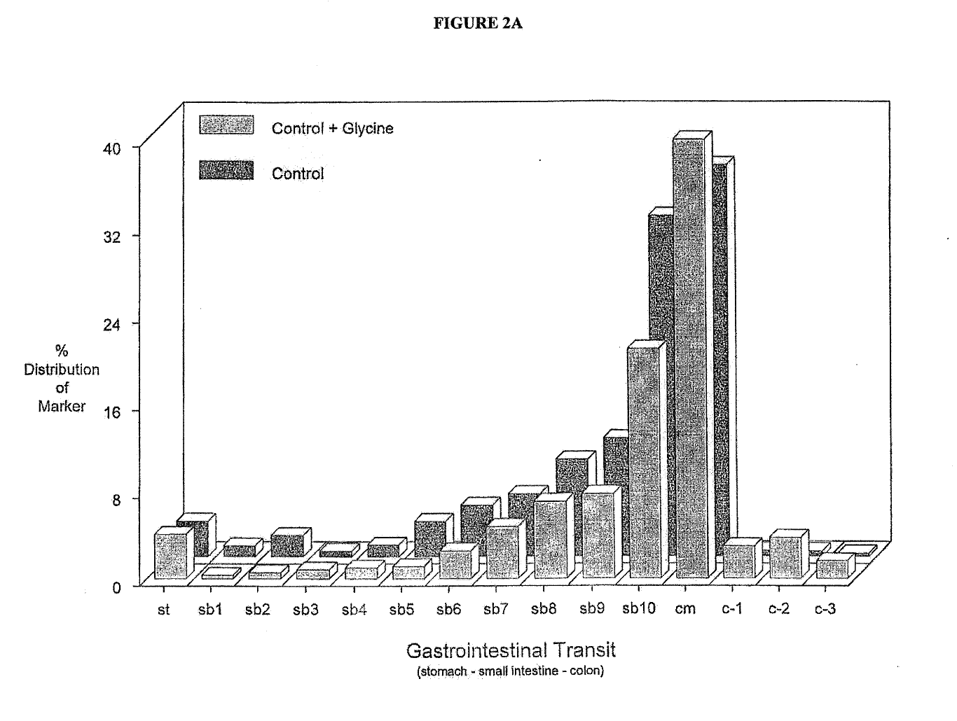

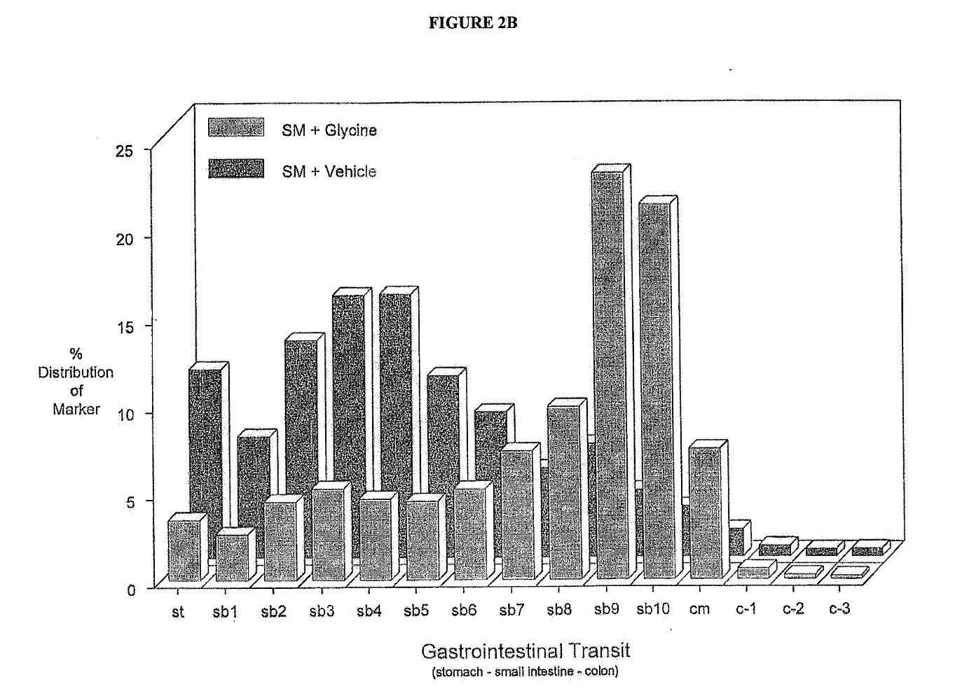

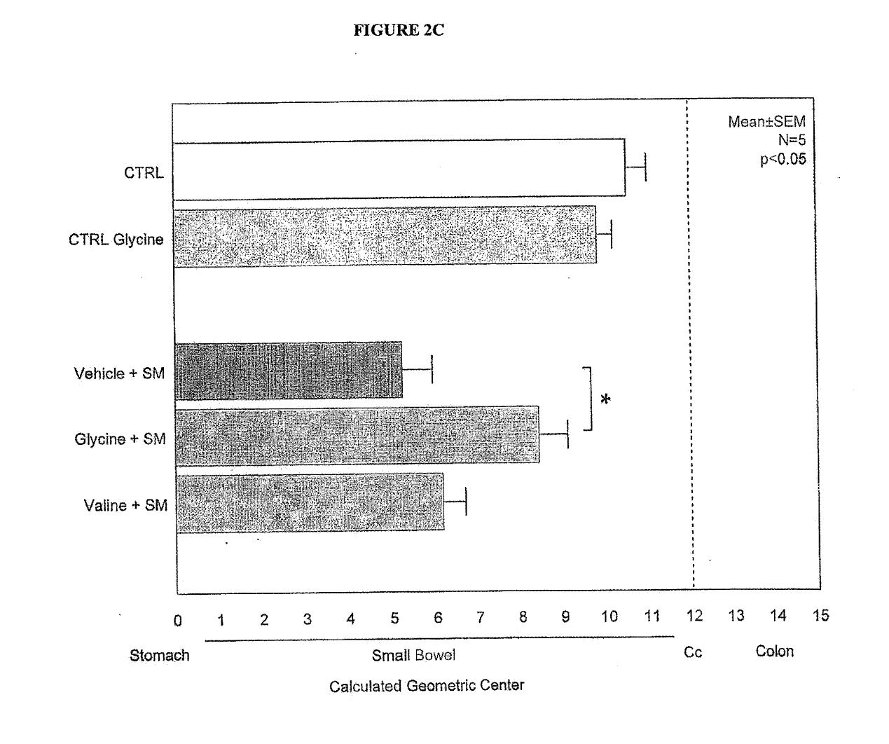

Glycine ameliorates postoperative muscle dysfunction: The potential of glycine pretreatment in preventing postoperative inflammatory dysmotility was analyzed in vivo by measuring gastrointestinal transit distribution histograms and in vitro using standard mechanical organ bath techniques. Postoperative changes in gastrointestinal motility were characterized using transit measurements and calculated geometric center analysis. As plotted in the histogram of FIG. 2A, non-manipulated vehicle and glycine treated rats presented with a similar distribution pattern of the non-absorbable transit marker two hours after oral administration (vehicle: GC=10.5±0.5; glycine: GC=9.8±0.4—FIG. 2C). Surgical manipulation caused a significant delay in intestinal transit among the vehicle-injected animals (GC=5.2±0.7). In this group, the fluorescence marker was retained within the proximal intestine yielding a delayed geometric center (FIGS. 2B and 2C). However, bowel manipulated rats that had received glycine (170 mg/kg) pretreatment before surgery showed a significantly improved postoperative transit (FIG. 2B) exhibiting a calculated geometric center of 8.5±0.7 (FIG. 2C). In contrast, the manipulated amino acid particle control pretreatment group demonstrated significant ileus with valine having no significant beneficial effect on postoperative gastrointestinal transit (GC=6.2±0.5), compared to manipulated vehicle-injected rats (FIG. 2C).

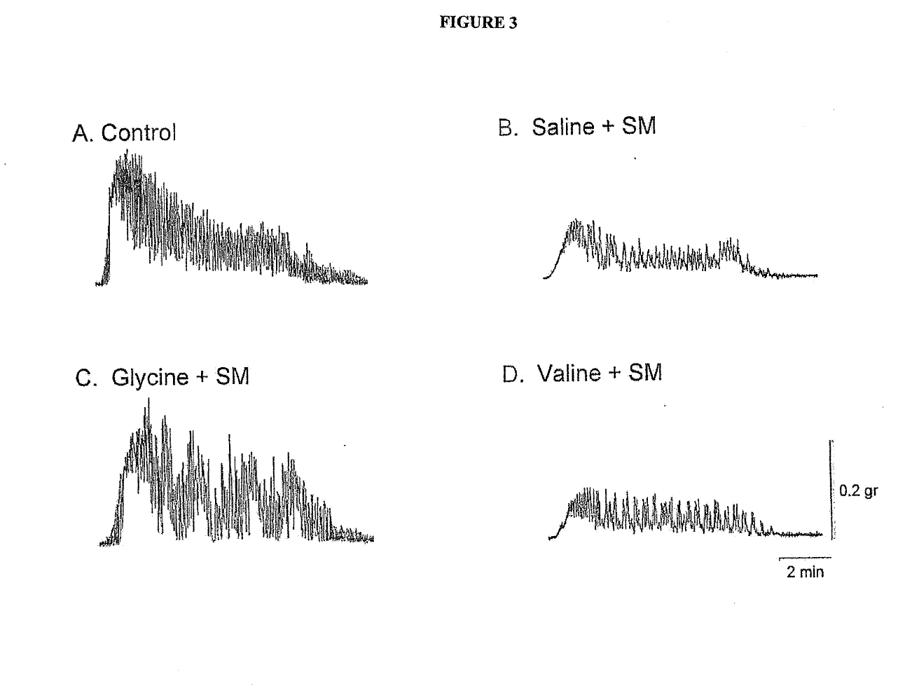

Next, we investigated the impact of glycine pretreatment directly to the jejunal and colonic muscularis by measuring in vitro spontaneous and bethanechol stimulated contractile activity. As illustrated in FIG. 3, bethanechol (100 mM) stimulated vehicle pretreated unmanipulated full thickness circular muscularis strips to generate a robust tonic contracture with overlying large phasic contractions. Intestinal manipulation caused a marked reduction of the contractile response to bethanechol and a near complete abolition of the tonic component. In contrast, glycine pretreatment attenuated the postoperative reduction in contractile activity (FIG. 3, Panel C), whereas valine pretreatment had no effect on the postoperative motor function of the circular muscularis (FIG. 3, Panel D).

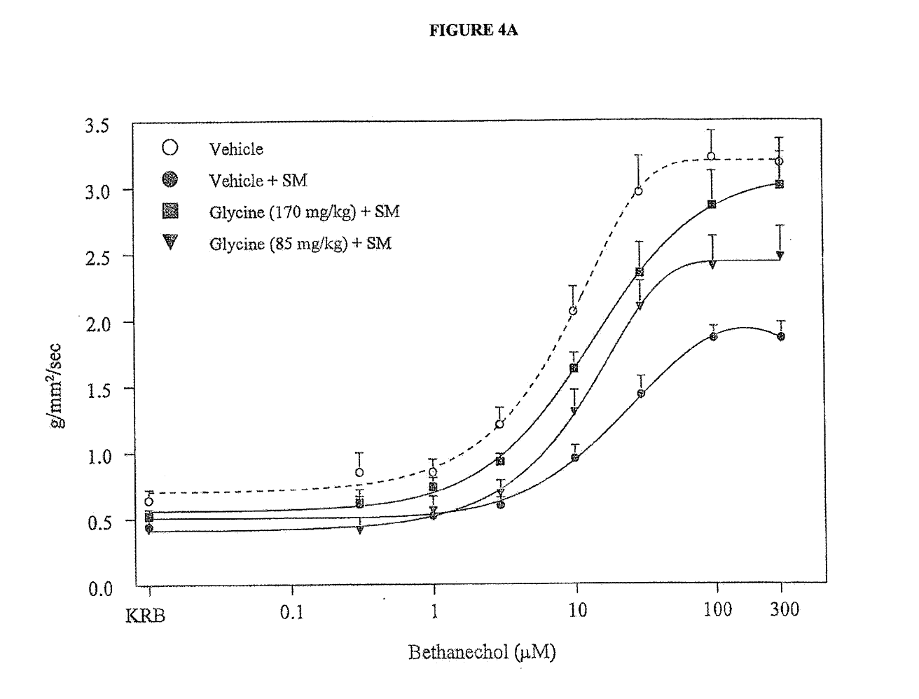

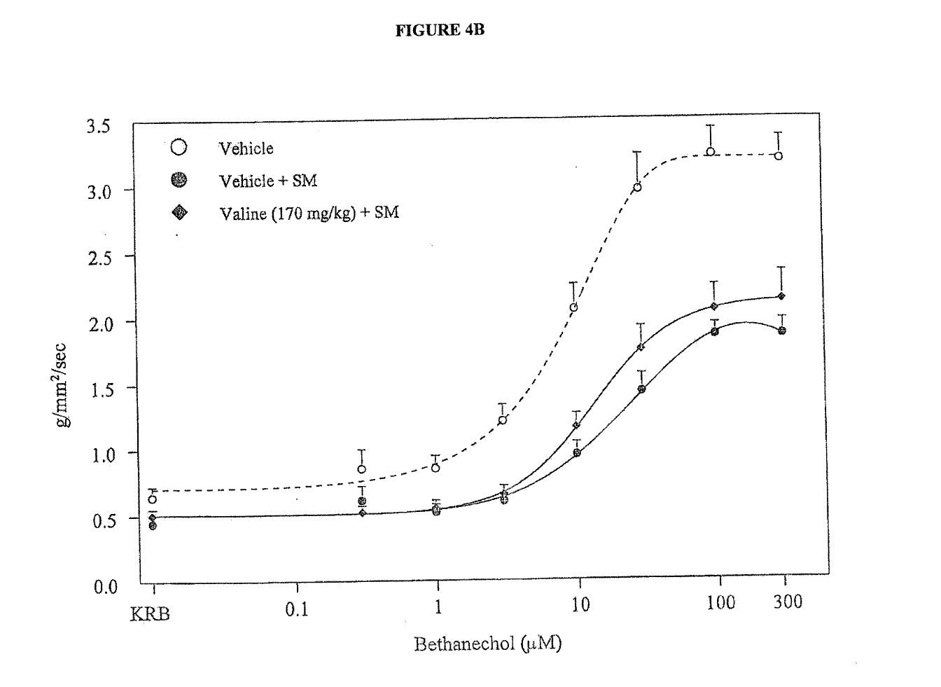

The direct effect of glycine 24 hour pretreatment to the jejunal muscularis externa was explored by generating complete bethanechol dose response curves for control, saline, glycine (85 mg/kg), glycine (170 mg/kg) and valine (170 mg/kg), as shown in FIGS. 4A and 4B. Jejunal circular muscles from non-manipulated, vehicle pretreated rats showed a progressive increase in the contractile force in response to increasing bethanechol stimulation and generated contractions with a mean contractile force of 3.2±0.2 g/mm2/s at 100 mM bethanechol. In contrast, intestinal manipulation caused a significant 42% reduction in the contractile responses (1.9±0.1 g/mm2/s at 100 mM). Muscle strips from manipulated glycine pretreated rats presented with a significant, dose dependent improvement in bethanechol stimulated contractile activity, compared to manipulated vehicle pretreated animals (85 mg/kg glycine: 2.4±0.3 g/mm2/s and 170 mg/kg glycine: 2.9±0.3 g/mm2/s at 100 mM bethanechol). And as illustrated in FIG. 4B, there was no significant difference in muscle contractility of manipulated valine pretreated animals (2.1±0.2 g/mm2/s at 100 mM) versus manipulated vehicle pretreated animals.

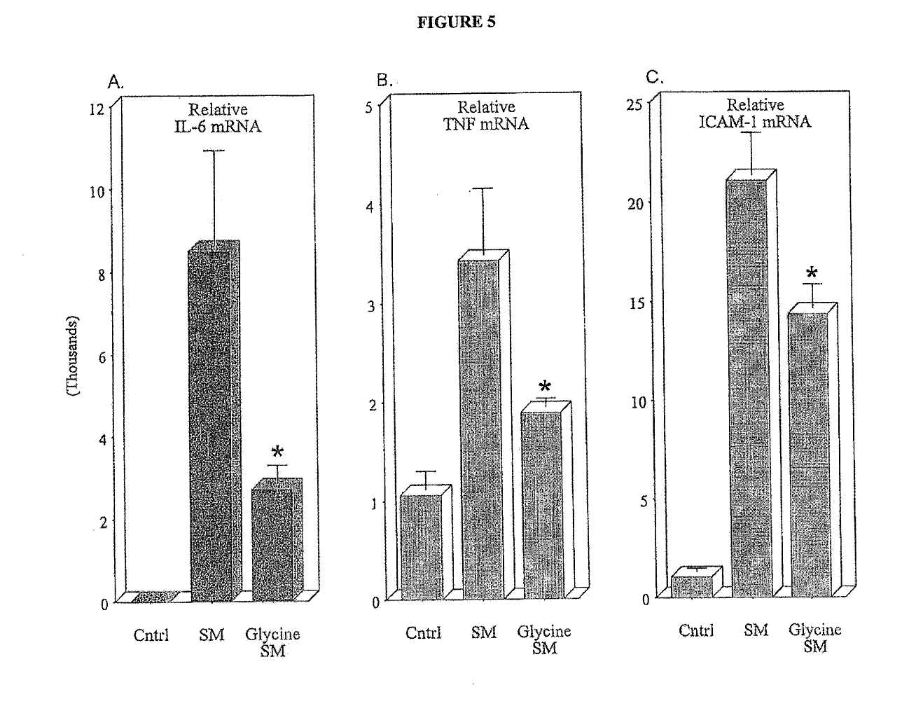

Glycine attenuates the molecular inflammatory response: We have previously shown, that early molecular inflammatory events are mechanistically involved in the postoperative recruitment of leukocytes into the intestinal muscularis (22), as well as the liberation of kinetically active substances (25,26). Thus, we investigated the impact of preoperative glycine treatment on the induction of prototypical inflammatory mediators in the early postoperative course. We focused on the mRNAs of the multifunctional cytokines IL-6 and TNF-a, and the leukocyte adhesion mediator ICAM-1. RT-PCR for IL-6 at 3 hours postoperatively in muscularis samples of the saline treated manipulated rats entered the exponential amplification phase significantly earlier than the saline controls demonstrating a higher template concentration (8521-fold). And as shown in FIG. 5A, glycine pretreated animals significantly attenuated the postoperative induction of IL-6 mRNA. A similar glycine attenuation of the postoperative induction of a molecular inflammatory response was also obtained for TNF-α and ICAM-1 mRNAs (FIGS. 5B and 5C). Mediator mRNA levels from non-operated vehicle rats did not differ from non-operated glycine injected animals and valine pretreatment of surgically manipulated animals had no effect on IL-6 mRNA levels.

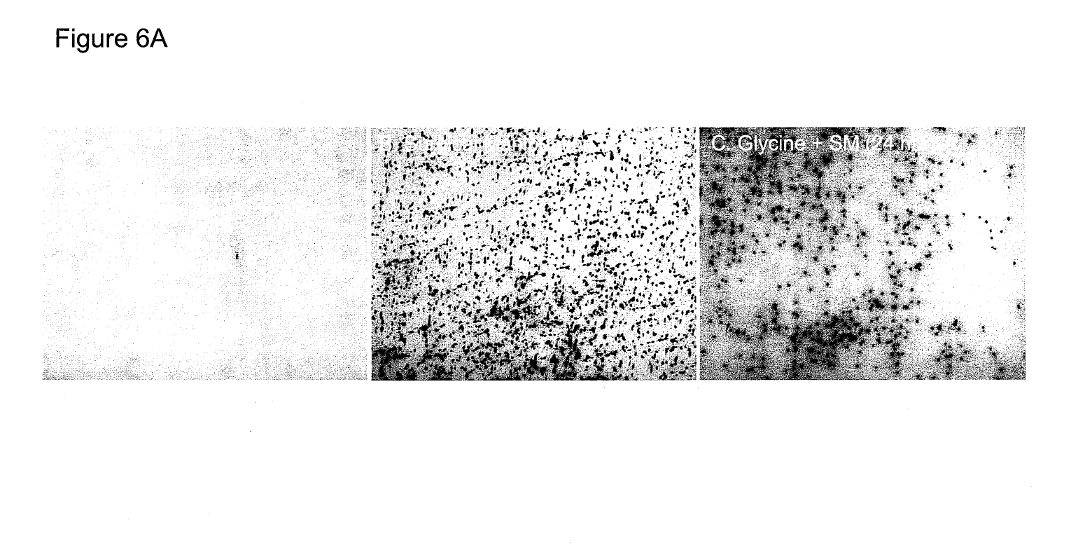

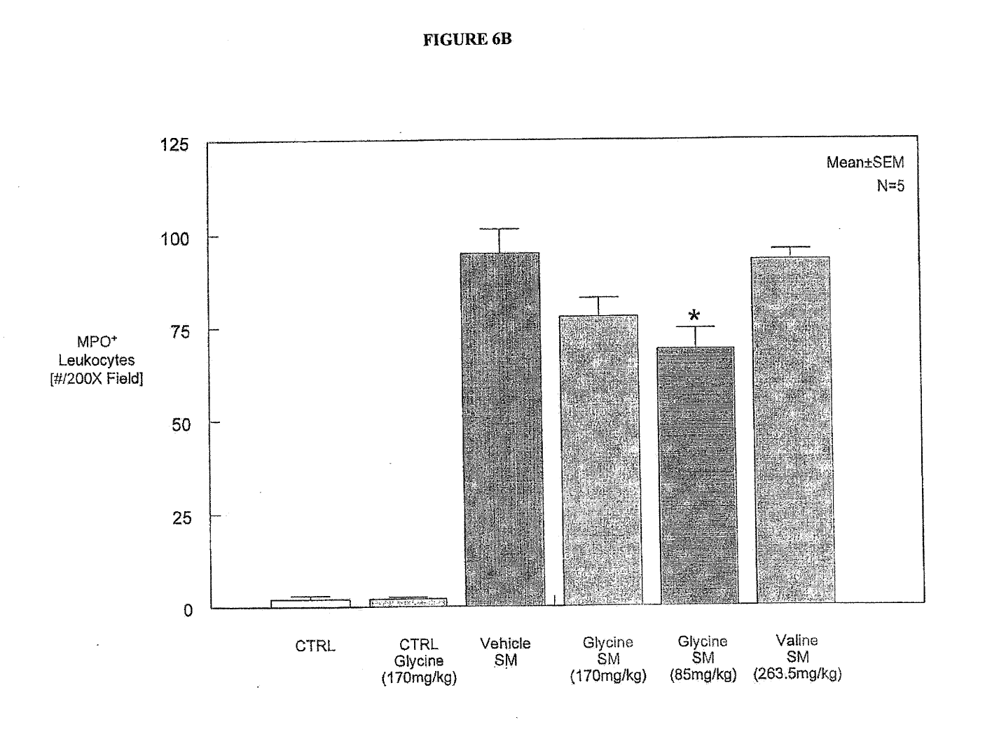

Glycine reduces postoperative leukocyte infiltrates within the intestinal muscularis: Based on the above results showing an attenuation in ICAM-1 mRNA and the knowledge that glycine potentially attenuates various postinjury inflammatory reactions in the liver and the lung (28,32,51), we hypothesized that preoperative exogenous glycine administration would reduce the postoperative recruitment of leukocytes into the manipulated small intestinal muscularis. Leukocyte infiltrates in the intestinal muscularis were characterized 24 hours postoperatively by myeloperoxidase (MPO) histochemistry. As illustrated in FIG. 6A and plotted in the histogram of FIG. 6B, vehicle or glycine injected rats without intestinal manipulation presented only with few MPO positive cells (vehicle: 2±0.9, glycine: 2±0.5 cells/field at 200× magnification). In accordance with our previous observations (14,22), FIG. 6A demonstrates that surgical manipulation of the small intestine results in a massive infiltration of neutrophils into the muscularis (95±6.4 cells/field at 200× magnification). However, as illustrated in (FIG. 6C) glycine pretreatment at 170 mg/kg resulted in a moderate but significant 27% reduction of infiltrating leukocytes compared to manipulated, vehicle pretreated rats (69±5.7 cells/field at 200×). Leukocyte infiltrates were significantly reduced by 18% in rats that had received 85 mg/kg glycine preoperatively (78±5.1 cells/field at 200×). In contrast, pretreatment with valine had no significant impact on the number of infiltrating cells in manipulated animals (93±2.7 cells/field at 200×) (FIG. 6B).

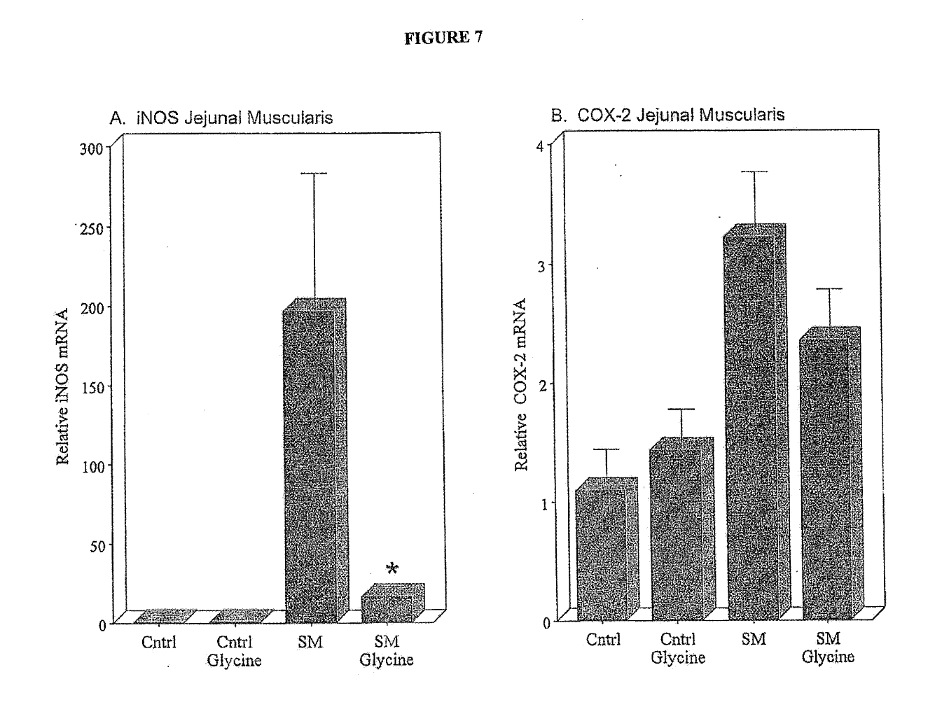

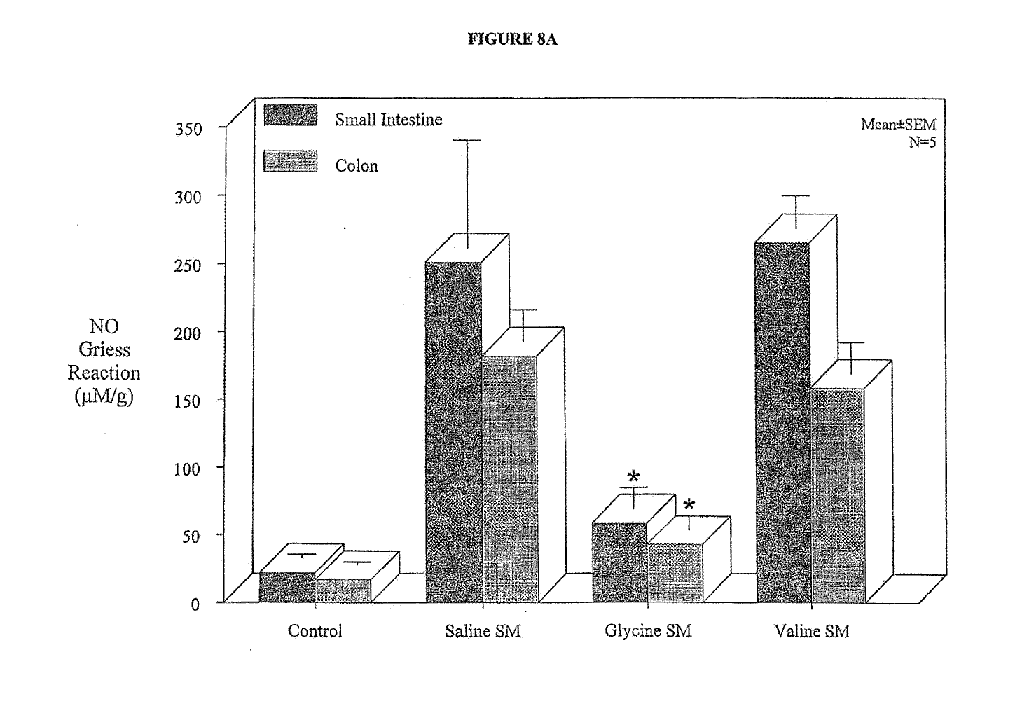

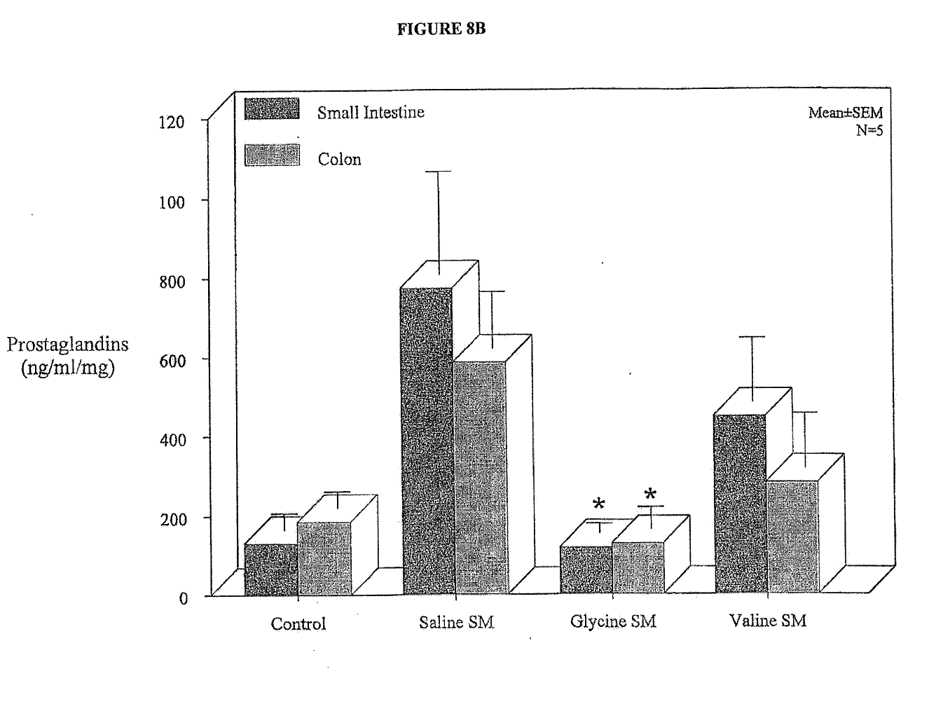

Glycine suppresses kinetically active mediators: We have previous shown the importance of the increased postoperative expression of the kinetically active mediator synthase iNOS and the leukocytic generation of nitric oxide in mediating postoperative ileus (52,53). In the present study, iNOS mRNA as upregulated 197.1-fold postoperatively at 3 hours compared to non-operated saline treated controls. This surgical induction was significantly suppressed by glycine pretreatment (FIG. 7A). We next investigated whether glycine pretreatment reduced the release of nitric oxide metabolites (nitrite) from the manipulated jejunal, and we also assayed their release from the isolate colonic muscularis as well. In accordance with our previous results 25, nitrite production was significantly increased by 11.4-fold and 10.9-fold in jejunal and colonic organ culture supernatants of manipulated vehicle pretreated rats compared to vehicle controls (FIG. 8A). While glycine (170 mg/kg) had no significant effects on Griess reaction measurements from the unmanipulated muscularis, the muscularis of rats that had received glycine (170 mg/kg) 1 hour before manipulation released significantly less nitrite compared to manipulated vehicle pretreated animals (2.7-fold and 2.5-fold increase over control for jejunum and colon). Valine pretreatment had no significant impact on nitrite release from the cultured manipulated muscularis of the jejunum or colon (12.0-fold and 9.5-fold, respectively).

Prostanoids produced through the increased expression of COX-2 have also been shown to be important in causing postoperative ileus (54,55). Indeed, the effect of glycine shown above for iNOS mRNA expression was also mimicked for COX-2 mRNA induction. As illustrated in FIG. 7B, COX-2 was significantly induced postoperative at 3 hours by surgical manipulation (3.2-fold). Where as, glycine pretreatment (170 mg/kg) tended to alleviate the postoperative upregulation (2.3 fold), this was not significant at this single time point by F-test with Bonferroni post-hoc group comparisons (FIG. 8B). However, we also measured prostaglandin release from the manipulated jejunal and colonic muscularis over a period of 24 hours in organ culture. As previously shown 54 and in concurrence with the postoperative upregulation in COX-2 mRNA, manipulation significantly increased prostanoid release into the culture media from the jejunal (5.9-fold) and colonic (3.1-fold) organ cultured muscularis (FIG. 8B). While the muscularis of rats that had received glycine (170 mg/kg) before manipulation released significantly less prostanoids over the 24 hours period of incubation compared to saline pretreated manipulated animals (0.9-fold and 0.7-fold for jejunum and colon). Although, there was a trend for valine pretreatment to decrease prostanoid release from the cultured manipulated muscularis of the jejunum and colon (3.4-fold and 1.5-fold increases from control, respectively), this was not significant. Mediator mRNA levels from non-operated vehicle rats did not differ from non-operated glycine injected animals (FIGS. 7A and 7B).

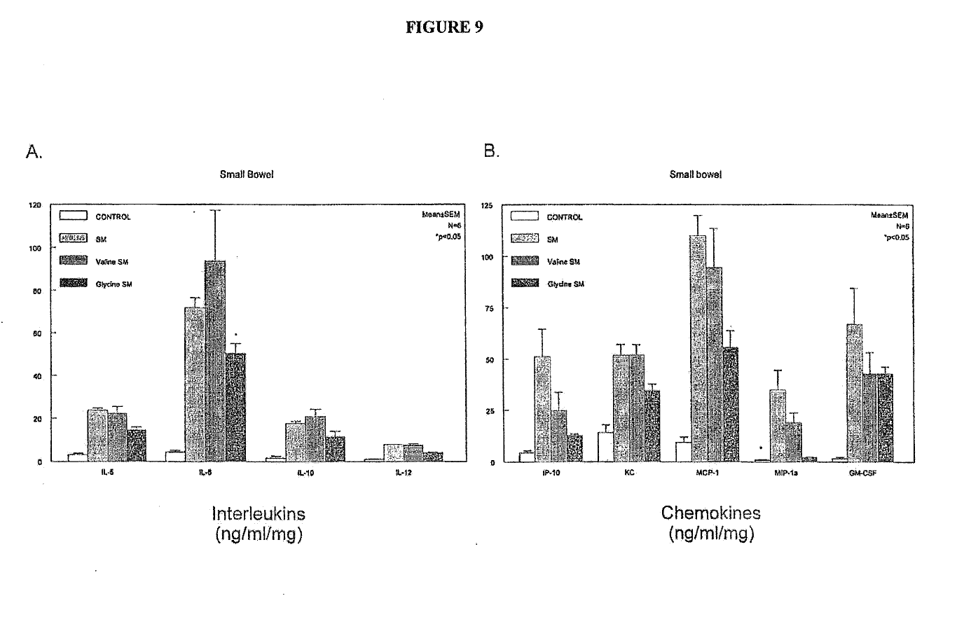

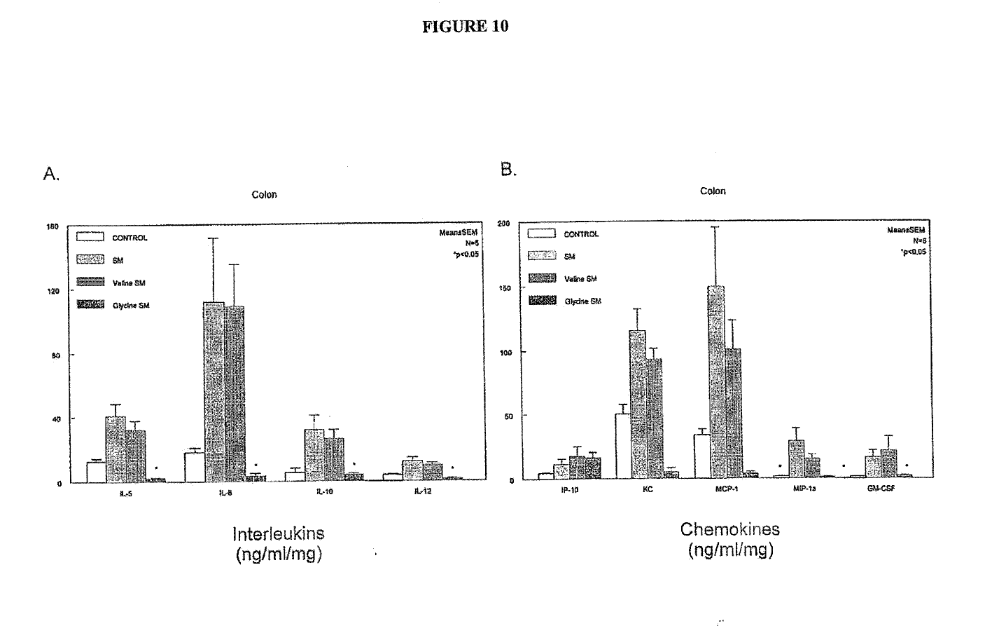

Glycine inhibits the release of interleukins and chemokines: A broad inhibition of the molecular inflammatory response was further investigated in C57Bl/6 mice, which also demonstrated a significant protection from the development of postoperative ileus by glycine (geometric centers: control=11.0±0.44, SM=4.1±0.18, SM+saline=4.1±0.5 and SM+glycine 170 mg/kg=10.3±7). Four groups of mice were analyzed using multiplex Luminex technology to measure the release of 20 inflammatory mediators in the media from the organ cultured muscularis of the jejunum and colon (FGF-basic, IL-1α, IL-1β, IL-2, IL-4, IL-5, IL-6, IL-10, IL-12p70, IL-13, IL-17, TNF-α, IFN-γ, GM-CSF, KC, MCP-1, MIG, MIP-1α, IP-10 and VEGF). As shown in FIG. 9A for the jejunum and FIG. 10A for the colon, glycine caused a decrease in the release of interleukins 5, 6, 10 and 12 with the response being more significantly pronounced in measurements from the colonic muscularis. A series of chemokines were also measured by the Luminex panel of inflammatory mediators (IP-10, KC, MCP-1, MIP-1α, VEGF and GM-CSF). These data shown in FIG. 9B and FIG. 10B demonstrate a trend for the inhibition of the production of these proteins by glycine with again the inhibition being more dramatic in the media from the colonic muscularis.

Conclusion: These data demonstrate that L-glycine protects against inflammatory postoperative ileus. Glycine pretreatment attenuated the molecular and cellular inflammatory events within the intestinal muscularis that occur after surgical manipulation of the intestine. Subsequently, postoperative alteration of smooth muscle contractile activity and delay in postoperative intestinal transit are minimized.

### REFERENCES

The following references may be referred to by number in this Example 3 in the preceding paragraphs:
1. Prasad M, Matthews J B. Deflating postoperative ileus. Gastroenterology 1999; 117:489-492.
2. Holte K, Kehlet H. Postoperative ileus: a preventable event. Br J Surg 2000; 87:1480-1493.
3. Livingston E H, Passaro E P, Jr. Postoperative ileus. Dig Dis Sci 1990; 35:121-132.
4. Delaney C P, Wolff B G, Viscusi E R et al. Alvimopan, for postoperative ileus following bowel resection: a pooled analysis of phase III studies[see comment]. Annals of Surgery 2007; 245:355-363.
5. Moss G, Regal M E, Lichtig L. Reducing postoperative pain, narcotics, and length of hospitalization. Surgery 1986; 99:206-210.
6. Chen H H, Wexner S D, Iroatulam A J et al. Laparoscopic colectomy compares favorably with colectomy by laparotomy for reduction of postoperative ileus. Dis Colon Rectum 2000; 43:61-65.
7. Piskun G, Kozik D, Rajpal S et al. Comparison of laparoscopic, open, and converted appendectomy for perforated appendicitis. Surg Endosc 2001; 15:660-662.
8. Maxwell-Armstrong C A, Robinson M H, Scholefield J H. Laparoscopic colorectal cancer surgery. Am J Surg 2000; 179:500-507.
9. Bardram L, Funch-Jensen P, Jensen P et al. Recovery after laparoscopic colonic surgery with epidural analgesia, and early oral nutrition and mobilisation. Lancet 1995; 345:763-764.
10. Basse L, Madsen L, Kehlet H. Normal gastrointestinal transit after colonic resection using epidural analgesia, enforced oral nutrition and laxative. Br J Surg 2001; 88:1498-1500.
11. Moss G. Maintenance of gastrointestinal function after bowel surgery and immediate enteral full nutrition. II. Clinical experience, with objective demonstration of intestinal absorption and motility. JPEN J Parenter Enteral Nutr 1981; 5:215-220.
12. Taguchi A, Sharma N, Saleem R M et al. Selective postoperative inhibition of gastrointestinal opioid receptors. N Engl J Med 2001; 345:935-940.
13. De Winter B Y, Boeckxstaens G E, De Man J G et al. Effect of different prokinetic agents and a novel enterokinetic agent on postoperative ileus in rats. Gut 1999; 45:713-718.
14. Kalff J C, Schraut W H, Simmons R L et al. Surgical manipulation of the gut elicits an intestinal muscularis inflammatory response resulting in postsurgical ileus. Ann Surg 1998; 228:652-663.
15. Hierholzer C, Kalff J C, Audolfsson G et al. Molecular and functional contractile sequelae of rat intestinal ischemia/reperfusion injury. Transplantation 1999; 68:1244-1254.
16. Hierholzer C, Kalff J C, Chakraborty A et al. Impaired gut contractility following hemorrhagic shock is accompanied by IL-6 and G-CSF production and neutrophil infiltration. Dig Dis Sci 2001; 46:230-241.
17. Kalff J C, Cicalese L, Exner B et al. Role of phagocytes in causing dysmotility after each stage of small bowel transplantation. Transplant Proc 1998; 30:2568.
18. Kalff J C, Buchholz B M, Eskandari M K et al. Biphasic response to gut manipulation and temporal correlation of cellular infiltrates and muscle dysfunction in rat. Surgery 1999; 126:498-509.
19. Eskandari M K, Kalff J C, Billiar T R et al. Lipopolysaccharide activates the muscularis macrophage network and suppresses circular smooth muscle activity. Am J Physiol 1997; 273:G727-G734.
20. Türler A, Schwarz N T, Türler E et al. MCP-1 causes leukocyte recruitment and subsequently endotoxemic ileus in rat. Am J Physiol Gastrointest Liver Physiol 2002; 282:G145-G155.
21. Kalff J C, Hierholzer C, Tsukada K et al. Hemorrhagic shock results in intestinal muscularis intercellular adhesion molecule (ICAM-1) expression, neutrophil infiltration, and smooth muscle dysfunction. Arch Orthop Trauma Surg 1999; 119:89-93.
22. Kalff J C, Carlos T M, Schraut W H et al. Surgically induced leukocytic infiltrates within the rat intestinal muscularis mediate postoperative ileus. Gastroenterology 1999; 117:378-387.
23. Josephs M D, Cheng G, Ksontini R et al. Products of cyclooxygenase-2 catalysis regulate postoperative bowel motility. J Surg Res 1999; 86:50-54.
24. Eskandari M K, Kalff J C, Billiar T R et al. LPS-induced muscularis macrophage nitric oxide suppresses rat jejunal circular muscle activity. Am J Physiol 1999; 277:G478-G486.
25. Kalff J C, Schraut W H, Billiar T R et al. Role of inducible nitric oxide synthase in postoperative intestinal smooth muscle dysfunction in rodents. Gastroenterology 2000; 118:316-327.
26. Schwarz N T, Kalff J C, Türler A et al. Prostanoid production via COX-2 as a causative mechanism of rodent postoperative ileus. Gastroenterology 2001; 121:1354-1371.
27. Rivera C A, Wheeler M D, Enomoto N et al. A choline-rich diet improves survival in a rat model of endotoxin shock. Am J Physiol 1998; 275:G862-G867.
28. Schemmer P, Bradford B U, Rose M L et al. Intravenous glycine improves survival in rat liver transplantation. Am J Physiol 1999; 276:G924-G932.
29. Spittler A, Reissner C M, Oehler R et al. Immunomodulatory effects of glycine on LPS-treated monocytes: reduced TNF-alpha production and accelerated IL-10 expression. FASEB J 1999; 13:563-571.
30. Thurman R G, Schemmer P, Zhong Z et al. Kupffer cell-dependent reperfusion injury in liver transplantation: new clinically relevant use of glycine. Langenbecks Arch Chir Suppl Kongressbd 1998; 115:185-190.
31. Wheeler M D, Rose M L, Yamashima S et al. Dietary glycine blunts lung inflammatory cell influx following acute endotoxin. Am J Physiol Lung Cell Mol Physiol 2000; 279:L390-L398.
32. Wheeler M D, Thurman R G. Production of superoxide and TNF-alpha from alveolar macrophages is blunted by glycine. Am J Physiol 1999; 277:L952-L959.
33. Wheeler M, Stachlewitz R F, Yamashina S et al. Glycine-gated chloride channels in neutrophils attenuate calcium influx and superoxide production. FASEB J 2000; 14:476-484.
34. Wheeler M D, Ikejema K, Enomoto N et al. Glycine: a new anti-inflammatory immunonutrient. Cell Mol Life Sci 1999; 56:843-856.
35. Fink L, Seeger W, Ermert L et al. Real-time quantitative RT-PCR after laser-assisted cell picking. Nat Med 1998; 4:1329-1333.
36. Kosuga K, Yui Y, Hattori R et al. Cloning of an inducible nitric oxide synthase from rat polymorphonuclear cells. Endothelium 1994; 2:217-221.
37. Xu K, Robida A M, Murphy T J. Immediate-early MEK-1-dependent stabilization of rat smooth muscle cell cyclooxygenase-2 mRNA by Galpha(q)-coupled receptor signaling. J Biol Chem 2000; 275:23012-23019.
38. Northemann W, Braciak T A, Hattori M et al. Structure of the rat interleukin 6 gene and its expression in macrophage-derived cells. J Biol Chem 1989; 264:16072-16082.
39. Kita Y, Takashi T, Iigo Y et al. Sequence and expression of rat ICAM-1. Biochim Biophys Acta 1992; 1131:108-110.
40. Yoshimura T, Takeya M, Takahashi K. Molecular cloning of rat monocyte chemoattractant protein-1 (MCP-1) and its expression in rat spleen cells and tumor cell lines. Biochem Biophys Res Commun 1991; 174:504-509.
41. Tso J Y, Sun X H, Kao T H et al. Isolation and characterization of rat and human glyceraldehyde-3-phosphate dehydrogenase cDNAs: genomic complexity and molecular evolution of the gene. Nucleic Acids Res 1985; 13:2485-2502.
42. Schmittgen T D, Zakrajsek B A, Mills A G et al. Quantitative reverse transcription-polymerase chain reaction to study mRNA decay: comparison of endpoint and real-time methods. Anal Biochem 2000; 285:194-204.
43. Kalff J C, Schwarz N T, Walgenbach K J et al. Leukocytes of the intestinal muscularis: their phenotype and isolation. J Leukoc Biol 1998; 63:683-691.
44. Green L C, Wagner D A, Glogowski J et al. Analysis of nitrate, nitrite, and [15N]nitrate in biological fluids. Anal Biochem 1982; 126:131-138.
45. Zhong Z, Wheeler M D, Li X et al. L-Glycine: a novel antiinflammatory, immunomodulatory, and cytoprotective agent. [Review][80 refs]. Current Opinion in Clinical Nutrition & Metabolic Care 2003; 6:229-240.
46. Turler A, Schnurr C, Nakao A et al. Endogenous endotoxin participates in causing a panenteric inflammatory ileus after colonic surgery. Annals of Surgery 2007; 245:734-744.
47. Eskandari M K, Kalff J C, Billiar T R et al. LPS-induced muscularis macrophage nitric oxide suppresses rat jejunal circular muscle activity. American Journal of Physiology—Gastrointestinal & Liver Physiology 1999; 277:G478-G486.
48. Overhaus M, Moore B A, Barbato J E et al. Biliverdin protects against polymicrobial sepsis by modulating inflammatory mediators. American Journal of Physiology—Gastrointestinal & Liver Physiology 2006; 290:G695-G703.
49. Hierholzer C, Kalff J C, Audolfsson G et al. Molecular and functional contractile sequelae of rat intestinal ischemia/reperfusion injury. Transplantation 1999; 68:1244-1254.
50. Hierholzer C, Kalff J C, Chakraborty A et al. Impaired gut contractility following hemorrhagic shock is accompanied by IL-6 and G-CSF production and neutrophil infiltration. Dig Dis Sci 2001; 46:230-241.
51. Schemmer P, Enomoto N, Bradford B U et al. Activated Kupffer cells cause a hypermetabolic state after gentle in situ manipulation of liver in rats. Am J Physiol Gastrointest Liver Physiol 2001; 280:G1076-G1082.
52. Kalff J C, Schraut W H, Billiar T R et al. Role of inducible nitric oxide synthase in postoperative intestinal smooth muscle dysfunction in rodents. Gastroenterology 2000; 118:316-327.
53. Turler A, Kalff J C, Moore B A et al. Leukocyte-derived inducible nitric oxide synthase mediates murine postoperative ileus. Annals of Surgery 2006; 244:220-229.
54. Schwarz N T, Kalff J C, Türler A et al. Prostanoid production via COX-2 as a causative mechanism of rodent postoperative ileus. Gastroenterology 2001; 121:1354-1371.
55. Kreiss C, Birder L A, Kiss S et al. COX-2 dependent inflammation increases spinal Fos expression during rodent postoperative ileus. Gut 2003; 52:527-534.
56. Lee M A, McCauley R D, Kong S E et al. Pretreatment with glycine reduces the severity of warm intestinal ischemic-reperfusion injury in the rat. Ann Plast Surg 2001; 46:320-326.
57. Iijima S, Shou J, Naama H et al. Beneficial effect of enteral glycine in intestinal ischemia/reperfusion injury. J Gastrointest Surg 1997; 1:53-60.
58. Schemmer P, Schoonhoven R, Swenberg J A et al. Gentle in situ liver manipulation during organ harvest decreases survival after rat liver transplantation: role of Kupffer cells. Transplantation 1998; 65:1015-1020.
59. Schemmer P, Bunzendahl H, Klar E et al. Reperfusion injury is dramatically increased by gentle liver manipulation during harvest. Transpl Int 2000; 13 Suppl 1:S525-S527.
60. Perera P Y, Vogel S N, Detore G R et al. CD 14-dependent and CD 14-independent signaling pathways in murine macrophages from normal and CD14 knockout mice stimulated with lipopolysaccharide or taxol. J Immunol 1997; 158:4422-4429.
61. Clapham D E. Calcium signaling. Cell 1995; 80:259-268.
62. Dolmetsch R E, Xu K, Lewis R S. Calcium oscillations increase the efficiency and specificity of gene expression. Nature 1998; 392:933-936.
63. Dolmetsch R E, Lewis R S, Goodnow C C et al. Differential activation of transcription factors induced by Ca2+ response amplitude and duration. Nature 1997; 386:855-858.
64. Ikejima K, Qu W, Stachlewitz R F et al. Kupffer cells contain a glycine-gated chloride channel. Am J Physiol 1997; 272:G1581-G1586.
65. Froh M, Thurman R G, Wheeler MD. Molecular evidence for a glycine-gated chloride channel in macrophages and leukocytes. American Journal of Physiology—Gastrointestinal & Liver Physiology 2002; 283:G856-G863.
66. Zhong Z, Enomoto N, Connor H D et al. Glycine improves survival after hemorrhagic shock in the rat. Shock 1999; 12:54-62.
67. Li X, Bradford B U, Wheeler M D et al. Dietary glycine prevents peptidoglycan polysaccharide-induced reactive arthritis in the rat: role for glycine-gated chloride channel. Infect Immun 2001; 69:5883-5891.
68. Romano M, Sironi M, Toniatti C et al. Role of IL-6 and its soluble receptor in induction of chemokines and leukocyte recruitment. Immunity 1997; 6:315-325.
69. Wortel C H, van Deventer S J, Aarden L A et al. Interleukin-6 mediates host defense responses induced by abdominal surgery. Surgery 1993; 114:564-570.
70. Ikejima K, Iimuro Y, Forman D T et al. A diet containing glycine improves survival in endotoxin shock in the rat. Am J Physiol 1996; 271:G97-103.
71. Mangino J E, Kotadia B, Mangino M J. Characterization of hypothermic intestinal ischemia-reperfusion injury in dogs. Effects of glycine. Transplantation 1996; 62:173-178.
72. Mauriz J L, Matilla B, Culebras J M et al. Dietary glycine inhibits activation of nuclear factor kappa B and prevents liver injury in hemorrhagic shock in the rat. Free Radic Biol Med 2001; 31:1236-1244.

### Example 4

This example demonstrates the obligatory recovery role of interleukin-10 in murine postoperative ileus.

### Methods

Animals. Adult male and female mice of 2-4 months of age weighing 25-30 grams were used. Homozygous 129P2/O1aHsd I110tm1Cgn/I110tm1Cgn backcrossed against C57BL/6 mice yielding B6.129P2-I110tm1Cgn/J mice (IL-10−/−) were obtained from Jackson Laboratory (Bar Harbor, Mass.). Wild-type C57BL/6 mice (IL-10+/+) were used as controls. The University of Pittsburgh Institutional Animal Care and Use Committee approved all experimental animal protocols. Animals were housed in a pathogen-free facility that is accredited by the American Association for Accreditation of Laboratory Animal Care and complies with the requirements of humane animal care as stipulated by the United States Department of Agriculture and the Department of Health and Human Services. They were maintained on a 12-hour light/dark cycle and provided with commercially available chow and tap water ad libitum.

Experimental groups and operative procedures. The small intestine of each IL-10+/+ and IL-10−/− animal was subjected to an easily standardized, surgical manipulation as described previously (6). In brief, before the beginning of surgery the animals were anesthetized by isoflurane inhalation and then placed on an isoflurane ventilator. Surgery consisted of a midline incision made into the peritoneal cavity followed by small bowel eventration to the left onto a moist gauze and manipulation of the entire small bowel by a rolling action between two moist cotton applicators. After manipulation the laparotomy was closed using a double layer running suture. The bowel manipulation procedure caused no mortality or bleeding. The animals recovered quickly from surgery and generally began to eat and drink within 6 hours. Animals were sacrificed at various time points between 0 and 7 days after manipulation. The animals were used to determine gastrointestinal transit, in vitro organ bath, isolated intestinal muscularis externa mRNA expression, protein analysis, nitric oxide and prostaglandin measurements, immunohistochemistry and histochemistry.

Gastrointestinal transit was measured in IL-10+/+ and IL-10−/− mice with and without intestinal surgical manipulation by evaluating the intestinal distribution of the aboral transit of the non-absorbable tracer, fluorescein isothiocyanate-labeled dextran (Invitrogen, Carlsbad, Calif.) with an average molecular mass of 70 kDa (FD70), as previously described (6, 14). Briefly, mice were orally fed 10 μl of FD70 dissolved in distilled water (6.25 mg/ml) under light anesthesia. Ninety minutes later, the animals were killed with an overdose of isoflurane inhalation. The entire gastrointestinal tract, from the lower esophageal sphincter to distal colon, was excised and divided into 15 segments: stomach, small intestine (10 segments of equal length), cecum, and colon (three segments of equal length). The lumenal contents of each segment was collected into a small tube and suspended in 800 μl of distilled water. The intestinal chyme in each tube was mixed vigorously and then clarified by centrifugation. The supernatants were collected and fluorometrically assayed for the FD70 concentration, which was then plotted into a gastrointestinal transit distribution histogram. Data from the distribution histogram was quantified and statistically analyzed by calculating the geometric center (GC) using the following formula; GC=Σ(% of total fluorescent signal per segment×segment number)/100 (15).

Mechanical activity of jejunal circular muscle strips was measured as previously described (16). A segment of mid-jejunum was pinned in a dissecting dish containing iced, preoxygenated KRB and then opened along the mesentery. The mucosa was stripped off and the muscularis was cut into strips (1×10 mm) parallel to the circular muscle layer and suspended in standard horizontal mechanical organ chamber. One end of each strip was tied to a fixed post and the other attached to an isometric force transducer (ADI, Colorado Springs, Colo.). In the organ chamber, each strip was allowed to equilibrate for one hour. Strips were then incrementally stretched to L0 (length at which maximal spontaneous contractions occur). Spontaneous activity and dose-response curves to increasing doses of bethanechol (0.3-300 μM) for 10 minutes with a 10 minute periods of KRB wash were generated from control and 24 hour postoperative manipulated muscle strips obtained from IL-10+/+ and IL-10−/− mice. Integrated muscle contractions for each 10 minute period were analyzed using the ADI analysis system (ADI, Colorado Springs, Colo.). Contractile activity was calculated as grams/mm2/sec by converting weight and length of the strip to square millimeters of tissue.

Total RNA was extracted from the isolated intestinal muscularis of mice at a series of specific time points after intestinal manipulation. The muscularis externa was isolated by cutting the entire small bowel into 5 cm segments, which were maintained in iced Krebs-Ringer Buffer (KRB). Each segment was then pinned down in a dissecting dish to remove the mesenteric tissue before it was slipped onto a glass rod. The muscularis was incised carefully along the mesentery and stripped off the mucosa by circumferentially peeling off the muscularis with a moist cotton applicator. The isolated muscularis was immediately snap frozen in liquid nitrogen and stored at −80° C. Total RNA extraction was performed as previously described (17), using the guanidinium thiocyanate phenol-chloroform extraction method. Two quantitative RT-PCR methods were used. TaqMan® Low Density Mouse Immune Panel using micro fluidic cards were used to quantify 96 TaqMan® Gene Expression targets known to have implications in immune response. Additionally, mRNA expressions for GAPDH, IL-6, IL-1β, CCL2 (MCP-1) and HO-1 were quantified in duplicate by SYBR Green two-step, real-time RT-PCR using GAPDH as the endogenous reference. Aliquots of stock RNA were subjected to first-strand complementary DNA (cDNA) synthesis using random hexamers (PE Applied Biosystems, Foster City, Calif.) and Super Script II (Life Technologies, Rockville, Md.). Primers were designed according to published sequences or from GenBank accession numbers using Primer Express software (PE Applied Biosystems, Foster City, Calif.). The following primer sequences for mouse were used:

| | | |
|---|---|---|
| IL-6 | Sense: 5′-TCA ATT CCA GAA ACC GCT ATG A-3′ | (SEQ ID NO: 13) |
| | Antisense: 3′-CAC CAG CAT CAG TCC CAA GA-5′ | (SEQ ID NO: 14) |
| IL-1β | Sense: 5′-CAG GTC GCT CAG GGT CAC A-3′ | (SEQ ID NO: 15) |
| | Antisense: 3′-CAG AGG CAA GGA GGA AAC ACA-5′ | (SEQ ID NO: 16) |
| CCL2 | Sense: 5′-CAA CTC TCA CTG AAG CCA GCT CT-3′ | (SEQ ID NO: 17) |
| | Antisense: 3′-CAG GCC CAG AAG CAT GAC A-5′ | (SEQ ID NO: 18) |
| HO-1 | Sense: 5′-CTC ACT GGC AGG AAA TCA TCC-3′ | (SEQ ID NO: 19) |
| | Antisense: 3′-ACC TCG TGG AGA CGC TTT ACA-5′ | (SEQ ID NO: 20) |
| GAPDH | Sense: 5′-TGA AGG TCG GTG TGA ACG GAT TTG GC-3′ | (SEQ ID NO: 21) |
| | Antisense: 3′-CAT GTA GGC CAT GAG GTC CAC CAC-5′ | (SEQ ID NO :22) |

The PCR reaction mixture was prepared using SYBR Green PCR Core Reagents (PE Applied Biosystems, Foster City, Calif.). Each sample was estimated in duplicate using the conditions recommended by the manufacturer. The reaction was incubated at 50° C. for 2 min to activate uracil N′-glycosylase, then at 95° C. for 10 min to activate Amplitaq Gold DNA polymerase, followed by 40 cycles of 95° C. for 15 sec and 60° C. for 1 min on an ABI PRISM 7700 Sequence Detection System (PE Applied Biosystems). Real-time PCR data were plotted as the ΔRn fluorescence signal versus the cycle number to determine the threshold cycle (CT). Quantification of mRNA expression was normalized to the GAPDH reference gene and calculated relative to control using the comparative CT method. To exclude PCR amplification of contaminating genomic DNA, RT-negative controls (samples containing RNA which were not reverse transcribed) were included in each PCR reaction. Gel electrophoresis was performed for the primers to confirm the absence of non-specific bands and that the amplicons were of the expected size. Efficiency of PCR-amplification of target cDNA was determined to assure colinearity of primer amplification. Standard curves were generated by plotting CT values against the relative input copy number. Slopes of −3.22±0.2 (r2=0.99) with corresponding efficiencies of 100±5% were considered acceptable. Melting curve analysis was performed for each PCR reaction to ensure amplification of a single product.

Inflammatory proteins produced by the isolated muscularis externa were determined using a Luminex assay. The small intestines of control and manipulated mice were removed under aseptic conditions. The small intestine was transferred to a sterile beaker containing DMEM culture medium with 200 U/ml penicillin G and 200 μg/ml streptomycin. The muscularis externa was isolated from the mucosa/lamina propria and aliquots of 70-100 mg tissue were organ cultured in 6 well culture plates containing 4 mls of serum free supplemented DMEM-culture medium in a CO₂-controlled incubator (NuAire, Plymouth, Minn.). The release of 20 inflammatory analytes into the tissue culture supernatant was quantified with a Luminex 100™ using microsphere-based multiplexing technology. The mouse Twenty-Plex immune kit was comprised of analyte specific components for the simultaneous measurement of the following mouse cytokines: FGF basic, GM-CSF, IFN-γ, IL-1α, IL-1β, IL-2, IL-4, IL-5, IL-6, IL-10, IL-12p40/p70, IL-13, IL-17, IP-10, KC, MCP-1, MIG, MIP-1α and TNF-α.

The release of nitric oxide (NO) and prostaglandins was also measured in organ culture media supernatant of the isolated muscularis externa obtained from IL-10+/+ and IL-10−/− mice without and with intestinal manipulation. The release of NO into the tissue culture supernatant over a period of 24 hours was assayed by a standard Griess reaction adapted to microplates, as described previously (18). The supernatant was frozen in liquid nitrogen and stored at −80° C. The muscle tissue was blotted dry and weighed. Griess reagent was prepared by mixing equal volumes of sulfanilamide (1.5% in 5% H₃PO₄) and naphthylethylenediamine dihydrochloride (0.1% in H₂O). A volume of 100 μl of reagent was mixed with 100 μl of supernatant and incubated at room temperature for 10 min. Absorbance of the formed chromophore was measured at 540 nm in an automated microplate reader. Nitrite was quantitated using NaNO₂ as a standard and results were expressed as μM nitrite/g tissue.

The synthesis of prostanoids released into the tissue culture supernatant over a period of 24 hours was assayed by ELISA. After organ culture incubation in the median, 500 μL aliquots of supernatant were frozen in liquid nitrogen and stored at −80° C. The muscle tissue was blotted dry and weighed. Culture supernatants were assayed for the measurement of prostaglandin secretion by enzyme immunoassay (Cayman Chemicals, Ann Arbor, Mich.). The ELISA was carried out in a 1:10 and 1:30 dilution. The assay was corrected for wet tissue weights and the prostaglandin ELISA kit sensitivity was 20 pg/ml.

Whole-mounts of the jejunal intestinal muscularis were investigated for the presence of resident and recruited myeloperoxidase-positive neutrophils and monocytes. Mid-jejunal segments were cut from the bowel and immersed in KRB in a chilled Sylgard® bottom covered glass dish as described previously 6. The length and width of each jejunal segment were measured with a caliper and then the segment was gently pinned down along the mesenteric border. The bowel was opened along the mesentery and stretched to 150% of the length and 250% of the width. The opened segments of jejunum were fixed in 100% ethanol for 10 min or 4% paraformaldehyde for 1 hour at room temperature or overnight at 40 C. Each segment was washed in three changes of KRB for 5 min each and the mucosa and submucosa were stripped off under microscopic observation (Leica MZ9-5, W. Nuhsbaum, Inc., McHenry, Ill.). Histological whole-mounts for staining were obtained from the mucosa-free muscularis by cutting each sheet of muscularis externa into 1×1 cm pieces. To visualize polymorphonuclear neutrophils, freshly prepared whole-mounts were subjected to a myeloperoxidase stain (10 mg Hanker-Yates reagent (Sigma, St. Louis, Mo.), 10 mL KRB and 100 μL 3% hydrogen peroxide) for 20 min at room temperature. The reaction was stopped by washing the whole-mounts in cold KRB. Muscularis whole-mounts were also processed for immunohistochemistry to detect resident and recruited macrophages/monocytes. Whole-mounts were blocked in 10% normal horse serum diluted in PBS (phosphate-buffered hypertonic saline, 1.8% NaCl in 0.01 M phosphate buffer, pH 7.4) containing 0.1% Triton X-100 and 10% normal horse serum for 1 hour at room temperature before incubation in primary antibody. Serotec F4/80 antibody conjugated to alexa-488 (1:200, rat polyclonal anti-mouse antibody, Harlan Bioproducts, Indianapolis, Ind.) was used to visualize macrophages/monocytes. All primary antibodies were diluted in antibody diluent. Whole-mounts were incubated in primary antibody at room temperature overnight. The specimens were then incubated in the appropriate secondary antibody (1:10,000, Cy3 donkey-anti-rabbit secondary antibody, Jackson Immunoresearch, West Grove, Pa.) at room temperature for 2 hours followed by three changes in PBS for 5 min each. Whole-mounts were cover slipped and inspected by light or fluorescent microscopy (Leica DMRX, W. Nushbaum, Inc., McHenry, Ill.). Leukocytes were counted in 5 randomly chosen areas in each specimen at a magnification of 200×. Nonspecific isotype-matched antibodies and primary antibody incubation without secondary antibody were used as negative controls.

Solutions and Statistics: A standard Krebs Ringers buffer (KRB) was used as described previously 17. This physiologic solution was gassed with 97% O2-3% CO₂ to establish a pH of 7.4. Muscle chamber temperatures were constantly monitored and maintained at 37±0.5° C. by the perfusion of the pre-warmed KRB solution. KRB constituents and bethanechol were obtained from Sigma Chemical Company (St. Louis, Mo.). Results are presented as means±standard error (SEM). The data were analyzed using student-t test or analysis of variance (ANOVA). EZAnalyze add-in for Microsoft Excel was used for F-test and Bonferroni post-hoc group comparisons where appropriate. p values<0.05 were considered significant.

### Results

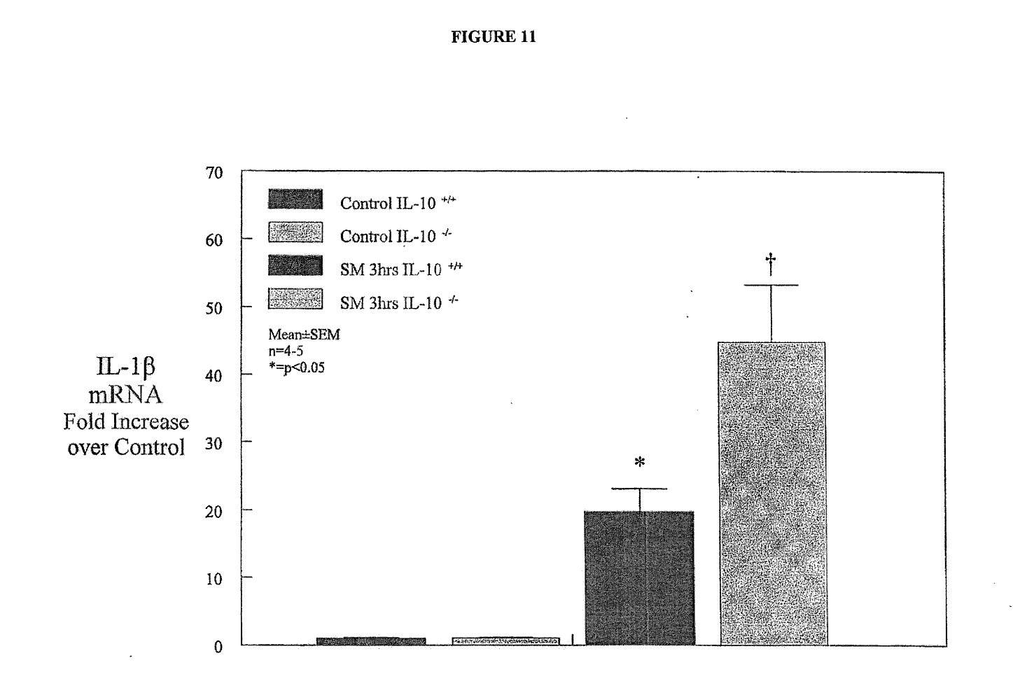

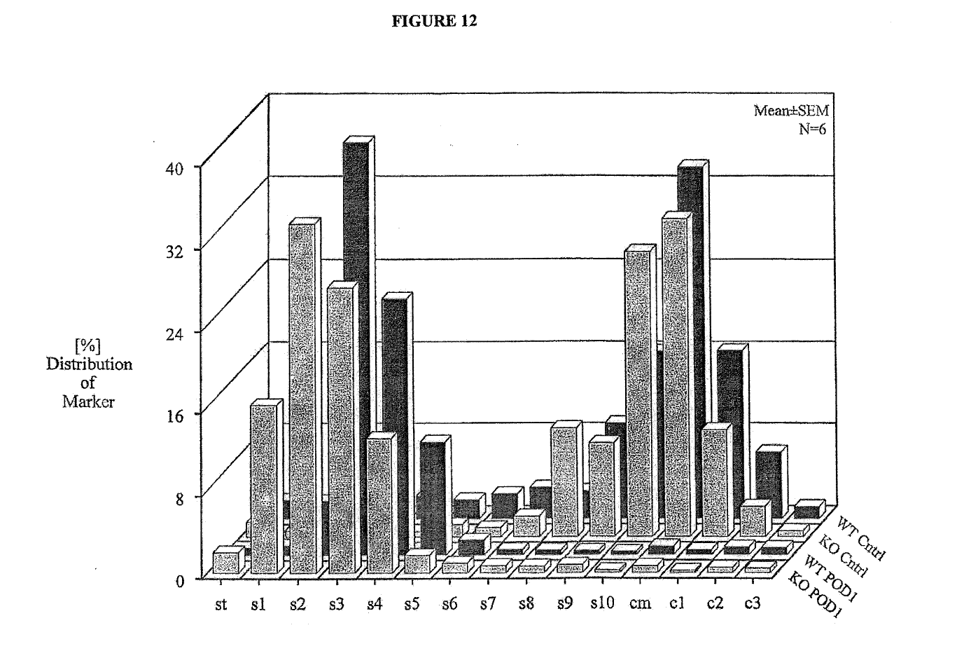

Early Postoperative TaqMan Array. We have previously shown that IL-10 mRNA is significantly, rapidly and persistently induced throughout a 24 hour time period following surgical manipulation of the intestine with peak induction occurring at 3 hours after surgery 6. Since IL-10 was induced in the rodent, we designed further studies in mouse to take advantage of the availability of the IL-10 knockout mouse on a C57Bl/6 background. Using the TaqMan® Low Density Array Mouse Immune Panel, we analyzed the expressions of 96 inflammatory mRNAs in isolated jejunal muscularis extracts of control and IL-10−/− animals without surgery or 3 hours after intestinal manipulation. As expected compared to C57Bl/6 control jejunal muscularis extracts, numerous inflammatory genes were significantly induced in the postoperative jejunal muscularis externa with greater than 3 fold inductions in NF-κB-1 and NF-κB-2, SOCS-1, MCP-1, MIP-1α, COX-2, HO-1, IL-6 and IL-1β determinations. Interestingly, in contrast to our expectation, similar levels of inductions were also observed in IL-10 knockout mice, except for a significantly enhanced induction in IL-1β mRNA (FIG. 11).

Early Postoperative Inflammatory Proteins by Luminex. We further looked for alterations in the production of numerous inflammatory proteins, which we have previously demonstrated are generated as part of the inflammatory milieu within the postsurgical muscularis externa. As above, four groups of mice were analyzed using multiplex Luminex technology to measure the release of 20 inflammatory mediators in the media from the organ cultured muscularis of the jejunum (control wild-type and IL-10−/− mice without surgery or 3 hours after surgical manipulation) (FGF-basic, IL-1α, IL-1β, IL-2, IL-4, IL-5, IL-6, IL-10, IL-12p70, IL-13, IL-17, TNF-α, IFN-γ, GM-CSF, KC, MCP 1, MIG, MIP-1α, IP-10 and VEGF). At this early time point after surgery in both wild-type and IL-10−/− mice, a significant increase in protein expressions were measured for GM-CSF, IL-6, KC, MCP-1 and MIP-1α (p<0.05, F-test with Bonferroni group comparisons). In concordance with the low density array, no significant differences were observed between the surgically manipulated wild-type and IL-10−/− mice (p>0.05).

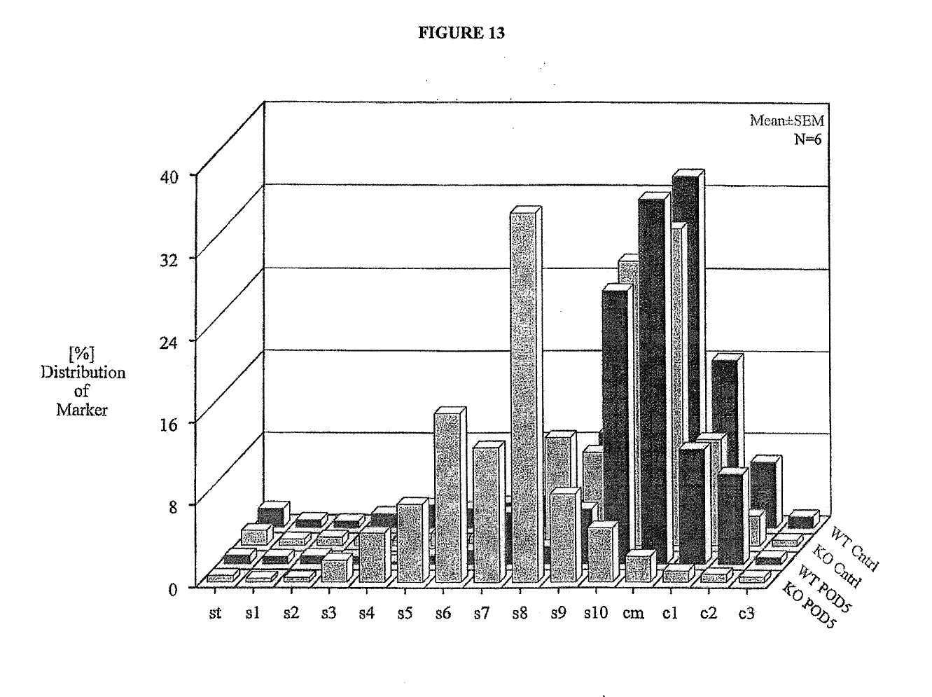

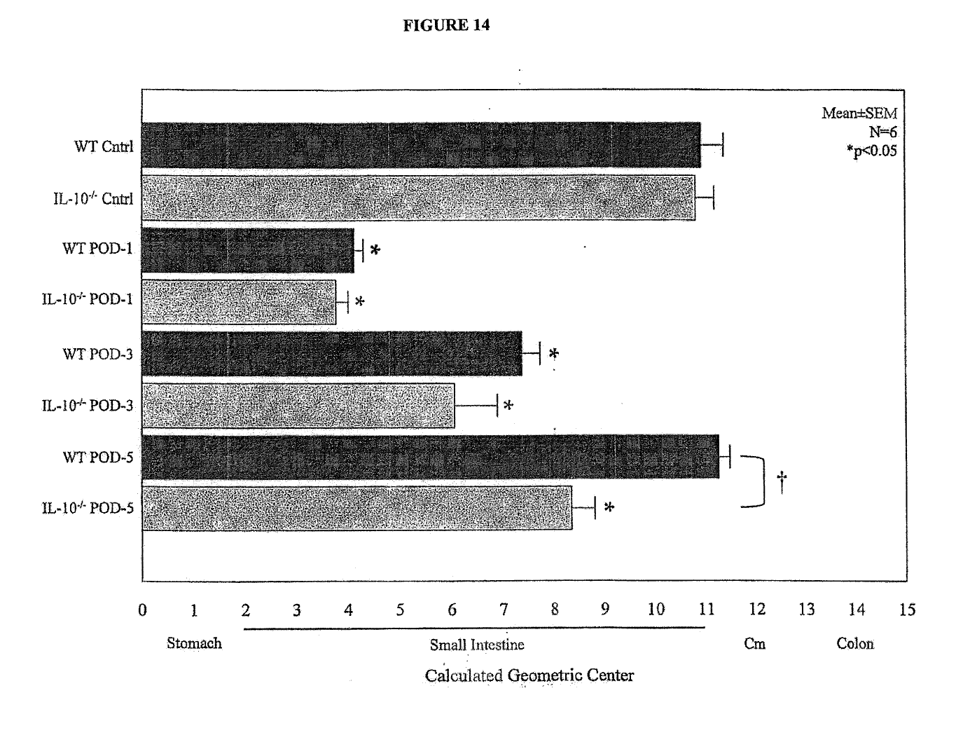

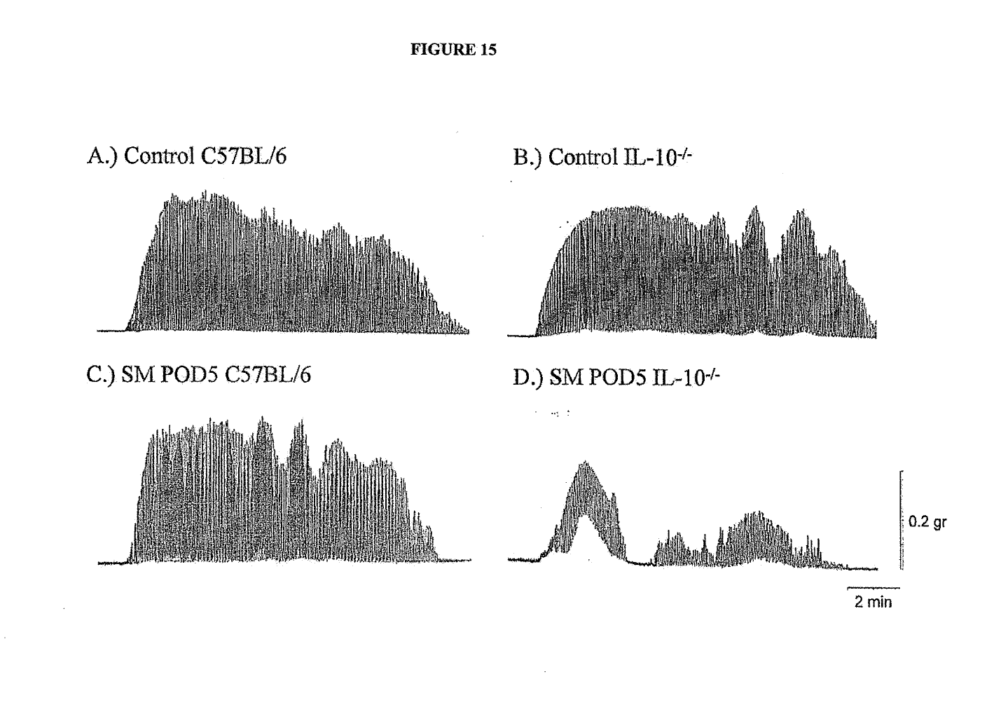

Postoperative Gastrointestinal Transit. The above molecular measurements in IL-10 knockout mice suggested that, although IL-10 is induced early after surgical manipulation of the intestine, the robust pro-inflammatory response is undeterred by this anti-inflammatory protein within the first hours after surgery. This conclusion was further confirmed by measuring intestinal function by generating gastrointestinal transit distribution histograms. In control and IL-10−/− mice, the orally administered, non-absorbable, FITC-dextran transit marker progressed to the distal segments of the intestine at the end of a 90 minute period (FIG. 13). As previously shown, surgical manipulation causes a significantly delay in gastrointestinal transit with the delay being similar when comparing IL 10+/+ and IL 10−/− mice (geometric center calculations—see FIGS. 14 and 15).

Postoperative ileus is a clinical problem that persists for a period of several days after surgery. We have previously shown in rat that postoperative jejunal muscle contractility recovers its normal function gradually over a period of approximately 7 days. Therefore, we sought to explore if IL-10 would play a crucial role in the recovery of the intestine from surgical manipulation. Gastrointestinal transit distribution histograms demonstrated in C57Bl/6 mice that on postoperative day 5 the aboral migration of the luminal transit marker was measured to be similar to unoperated animals, hence recovery from the surgical trauma had taken place (FIG. 14). In contrast, gastrointestinal transit in the IL-10−/− mice remained significantly delayed on postoperative day 5 with much of the transit marker located in the middle of the jejunum after the 90 minute period of motility before sacrifice. FIG. 14 shows the calculated geometric center values for groups of C57Bl/6 and IL-10−/− mice without operation and then for animals 1, 3 and 5 days after surgical manipulation with a significant difference between C57Bl/6 and IL-10−/− mice on postoperative day 5 with a lagging trend in transit seen already at postoperative day 3. We sought to comparatively measure gastrointestinal function in the two genetic backgrounds on postoperative day 7, and as expected transits were normal in manipulated C57Bl/6 mice (geometric center=10.9±0.24). But, dramatically, 7 of 8 mice with an IL-10−/− genetic background died by the 7th postoperative day with no mortality observed in the wild-types. Hence, IL-10 appeared obligatory for recovery of the animal from the surgical insult.

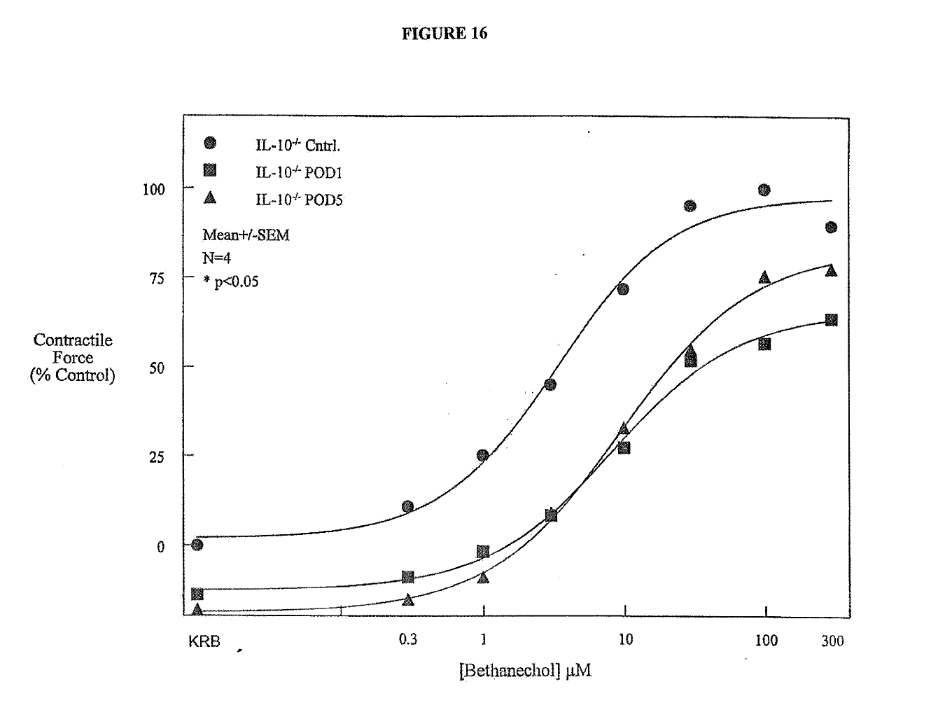

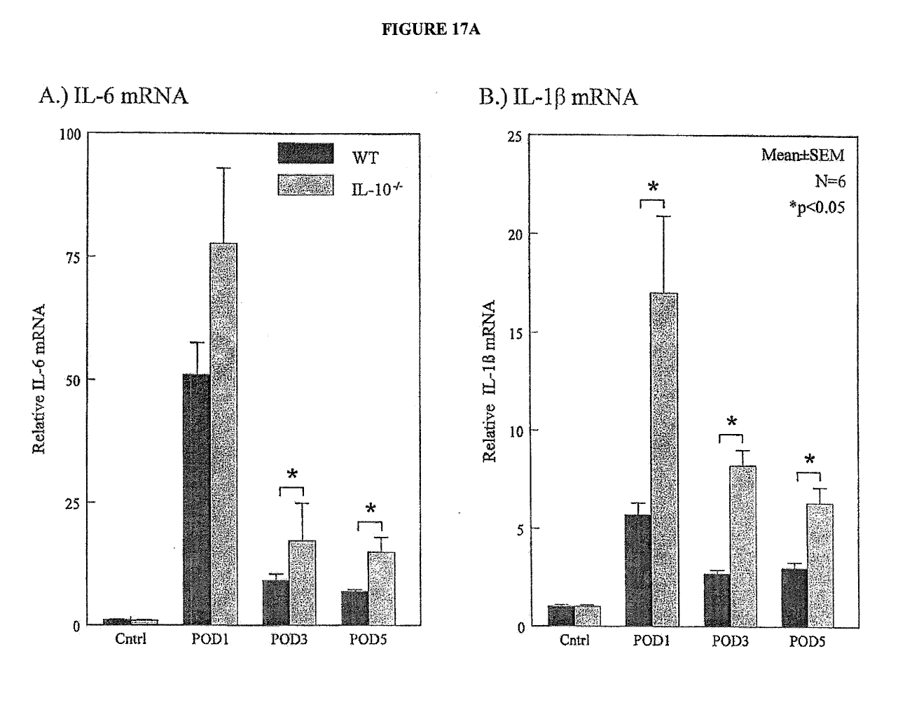

Postoperative Jejunal Muscle Contractility. Experiments were next designed to investigate the specific obligatory role of IL-10 directly on the recovery of jejunal circular smooth muscle contractility. Muscle strips were prepared from un manipulated controls and manipulated mice on postoperative days 1 and 5 with the distinct genetic backgrounds. As previously published and, therefore, not shown here, surgical manipulation of wild-type rodents resulted in a significant suppression of jejunal circular muscle contractions to bethanechol stimulation. As shown in FIG. 16, Panels A and C, wild-type rodent jejunal muscle contractility to bethanechol (100 μM) was well recovered after a period of 5 days. Although, un-manipulated muscles from IL-10−/− mice developed a robust contractile response to bethanechol (FIG. 6, Panel B), this response remained markedly diminished in muscles obtained from the manipulated IL-10−/− mice on postsurgical day 5 (FIG. 6, Panel D). Data illustrated in FIG. 7 delineates the complete dose response curves for bethanechol stimulated jejunal circular muscles of L-10−/− mice taken from unoperated controls and surgically manipulated animals on postoperative days 1 and 5. Circular muscle contractions were diminished all along the dose response curve after surgery and the contractile activity never recovered, as IL-10−/− mice died on postoperative day 7.

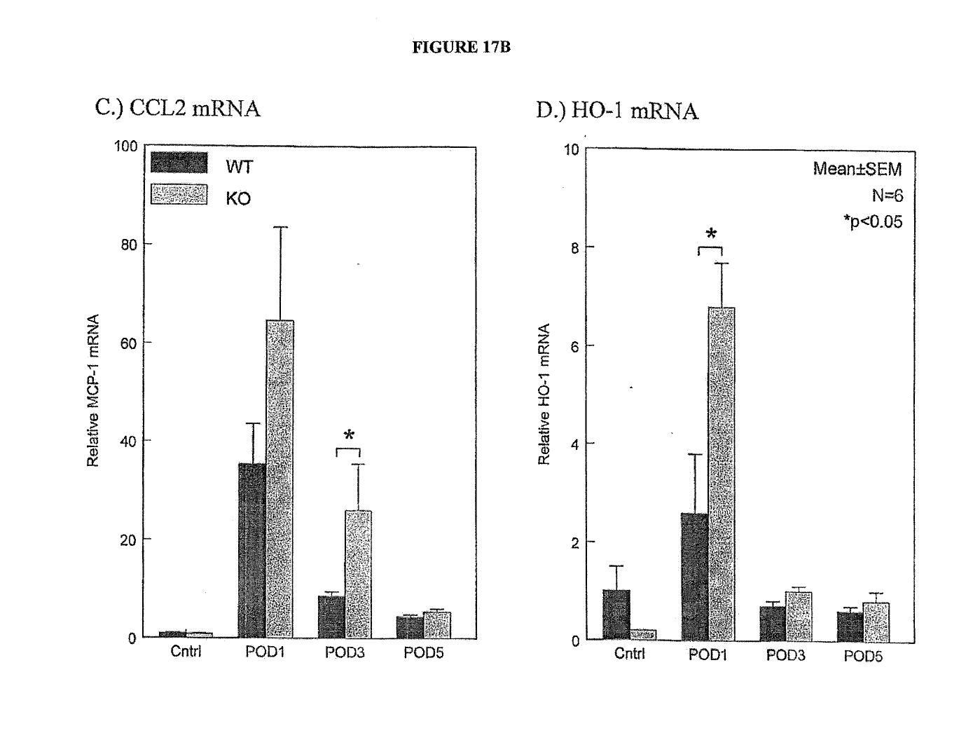

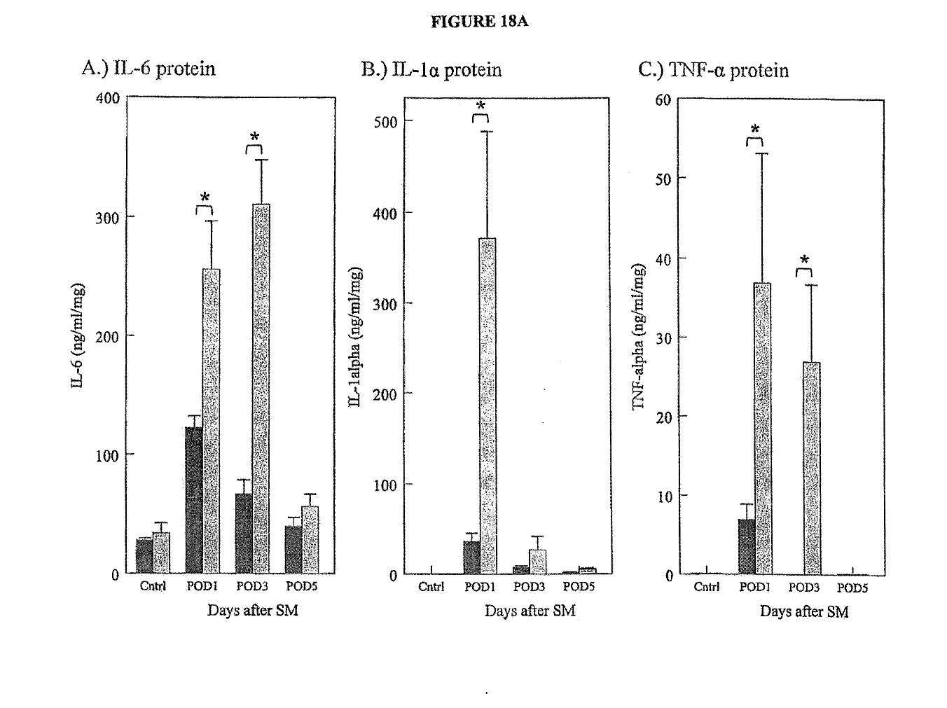

Late Postoperative Inflammatory mRNAs by qPCR. The above functional data indicated that IL-10 was playing a role in the recovery phase of postoperative ileus, so we investigated the expression of inflammatory mediators at later time points by RT-PCR on postoperative days 1, 3 and 5, as at 3 hours the initiating inflammatory mRNAs analyzed by the TaqMan® Low Density Array Mouse Immune Panel did not show significant differences between wild-type and IL-10 knockout mice, except for IL-1β. As shown in the mRNA histograms in FIG. 17, Panels A-C, the mRNAs of the prototypical cytokines IL 6, IL-1β and MCP-1 demonstrated increased surgical induction in the IL-10−/− mice compared to wild-type mice at various time points (*=p>0.05, F-test with Bonferroni group comparisons, N=5 each). Additionally, mRNA of the anti-inflammatory mediator HO-1 was significantly induced to a greater magnitude in IL-10−/− 24 hours after surgical manipulation compared to IL-10+/+ mice at the same time (FIG. 17, Panel D).

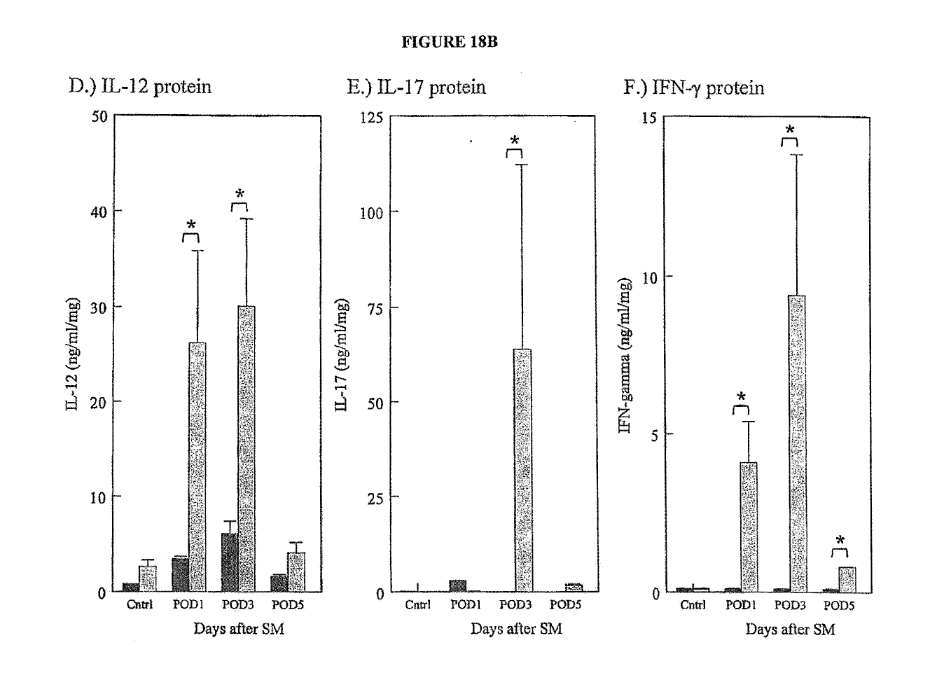

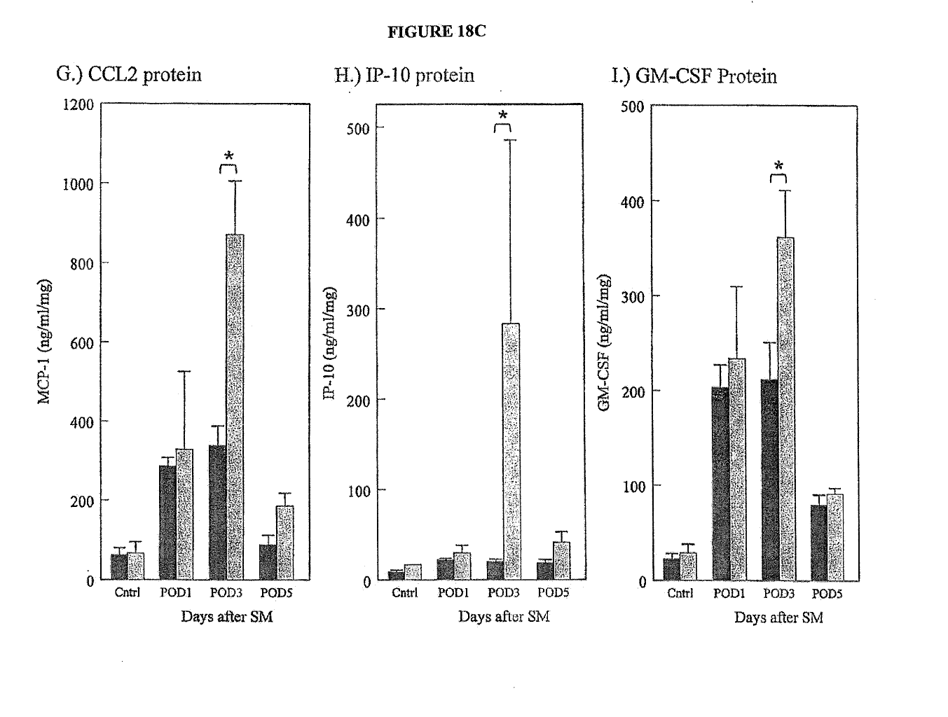

Late Postoperative Inflammatory Proteins by Luminex. The production of an array of inflammatory proteins generated by the isolated muscularis externa was next investigated using a mouse Twenty-Plex Luminex assay. The intestinal muscularis was isolated from unoperated and surgically manipulated mice at 0, 1, 3 and 5 postoperative days obtained from both control wild-type C57Bl/6 and IL-10−/− mice, which was then incubated for a 24 hour period in 4 ml DMEM in an oxygenated culture chamber (N=5 each). The tissue weight adjusted release of twenty mouse cytokines was determined in the supernatant of each incubated tissue sample (FGF basic, GM-CSF, IFN-γ, IL-1α, IL-1β, IL-2, IL-4, IL-5, IL-6, IL-10, IL-12p40/p70, IL-13, IL-17, IP-10, KC, MCP-1, MIG, MIP-1α and TNF-α). No significant difference in baseline release of these inflammatory mediators was measured in the twenty analytes of unoperated wild-type and unoperated IL-10−/− mice, indicating no pre-existing basal inflammatory response in the IL-10−/− mouse small intestine as in the above 3 hour postoperative Luminex assay. In correlation with the RT-PCR measurements, Luminex analysis of 20 analytes showed significant increases in interleukins (IL-6, IL-1α, IL-12, IL-17), TNF-α and chemokines (MCP-1, IP-10 and GM-CSF) on multiple days after surgery in the IL-10−/− mice compared to wild-type mice (FIG. 18, Panels A-I).

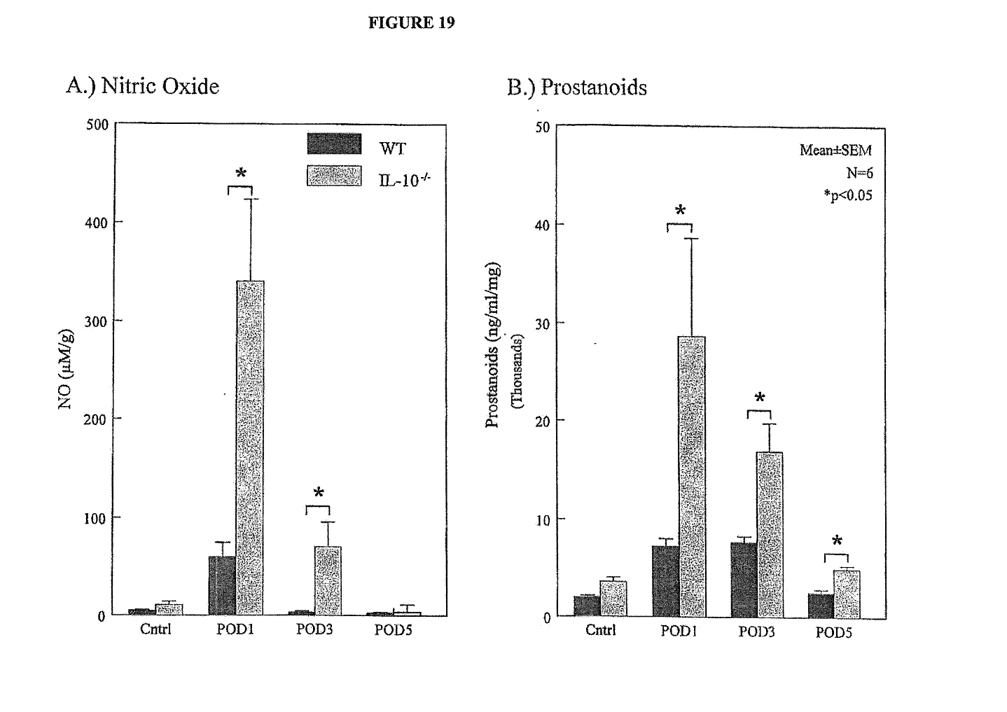

Late Kinetic Mediators. In addition to inflammatory cytokines and chemokines, we have shown that nitric oxide and prostanoids play a key role in directly suppressing postoperative gastrointestinal motility. Therefore, we investigated if these two kinetic factors were also dependent on IL-10. As illustrated in FIG. 19, Panel A, nitric oxide released from the organ cultured muscularis for a 24 hour period after harvest on day 1 was significantly increased by 12.9 fold in surgically manipulated wild-type mice on postoperative day 1 compared to un-manipulated wild-type muscularis. In IL 10−/− mice nitric oxide levels measured by Griess reaction were a significant 5.6 and 23.7 fold greater compared to wild-type on postoperative days 1 and 3.

In addition to nitric oxide, prostanoid release from the inflamed IL-10−/− muscularis was also increased (FIG. 19, Panel B). First, as we have previously reported in wild-type mice, bowel manipulation significantly increases the release of prostanoids from the manipulated muscularis harvested on postoperative day 1 (3.7 fold) and as seen here this persists through at least postoperative day 3 (4.0 fold). In IL-10 knockout mice, this increase in prostanoid production by the isolated muscularis externa was significantly 3.9, 2.2 and 2.0 fold greater on postoperative days 1, 3 and 5, respectively, compared to wild-types (p<0.05).

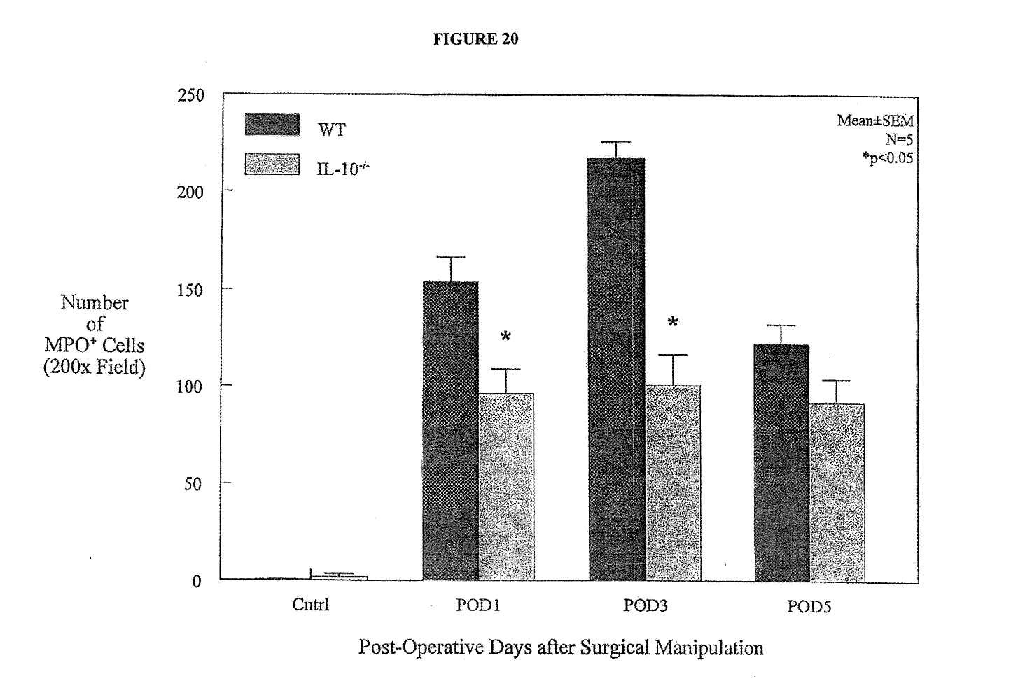

We have shown previously that the inflammatory cytokines, chemokines, nitric oxide and prostanoids are primarily secreted by the resident macrophage network and the infiltrating neutrophils and monocytes after surgery. Indeed, in wild-type mice and IL-10−/− mice, anesthesia and surgical manipulation of the intestine resulted in a significant increase in the extravasation of both neutrophils into the manipulated jejunal muscularis (FIG. 20). But, unexpectedly, the absolute numbers of neutrophils recruited into the manipulated muscularis of the IL-10−/− mice was significantly less compared to controls.

### REFERENCES

The following references may be referred to by number in this Example 4 in the preceding paragraphs:
1. Delaney C P, Wolff B G, Viscusi E R, Senagore A J, Fort J G, Du W, Techner L, Wallin B. Alvimopan, for postoperative ileus following bowel resection: a pooled analysis of phase III studies[see comment]. Annals of Surgery 2007; 245:355-363.
2. Bauer A J, Boeckxstaens G E. Mechanisms of postoperative ileus. Neurogastroenterology & Motility 2004; 16: Suppl-60.
3. Kalff J C, Schraut W H, Billiar T R, Simmons R L, Bauer A J. Role of inducible nitric oxide synthase in postoperative intestinal smooth muscle dysfunction in rodents. Gastroenterology 2000; 118:316-327.
4. Schwarz N T, Kalff J C, Turler A, Engel B M, Watkins S C, Billiar T R, Bauer A J. Prostanoid production via COX-2 as a causative mechanism of rodent postoperative ileus. Gastroenterology 2001; 121:1354-1371.
5. Turler A, Kalff J C, Moore B A, Hoffman R A, Billiar T R, Simmons R L, Bauer A J. Leukocyte-derived inducible nitric oxide synthase mediates murine postoperative ileus. Annals of Surgery 2006; 244:220-229.
6. Moore B A, Otterbein L E, Türler A, Choi A M, Bauer A J. Inhaled carbon monoxide suppresses the development of postoperative ileus in the murine small intestine. Gastroenterology 2003; 124:377-391.
7. Moore B A, Overhaus M, Whitcomb J, Ifedigbo E, Choi A M, Otterbein L E, Bauer A J. Brief inhalation of low-dose carbon monoxide protects rodents and swine from postoperative ileus. Critical Care Medicine 2005; 33:1317-1326.
8. Murray P J. Understanding and exploiting the endogenous interleukin-10/STAT3-mediated anti-inflammatory response. Current Opinion in Pharmacology 2006; 6:379-386.
9. Otterbein L E, Bach F H, Alam J, Soares M, Lu H T, Wysk M, Davis R J, Flavell R A, Choi A M. Carbon monoxide has anti-inflammatory effects involving the mitogen-activated protein kinase pathway. Nature Medicine 2000; 6:422-428.
10. Tamion F, Richard V, Renet S, Thuillez C. Protective effects of heme-oxygenase expression against endotoxic shock: inhibition of tumor necrosis factor-alpha and augmentation of interleukin-10. Journal of Trauma-Injury Infection & Critical Care 2006; 61:1078-1084.
11. Wirtz S, Neufert C, Weigmann B, Neurath M F. Chemically induced mouse models of intestinal inflammation. Nature Protocols 2007; 2:541-546.
12. Barbara G, Xing Z, Hogaboam C M, Gauldie J, Collins S M. Interleukin 10 gene transfer prevents experimental colitis in rats. Gut 2000; 46:344-349.
13. Bickston S J, Cominelli F. Recombinant interleukin 10 for the treatment of active Crohn's disease: lessons in biologic therapy[comment]. Gastroenterology 2000; 119:1781-1783.
14. Moore B A, Turler A, Pezzone M A, Dyer K F, Grandis J R, Bauer A J. Tyrphostin AG 126 inhibits development of postoperative ileus induced by surgical manipulation of murine colon. Am J Physiol Gastrointest Liver Physiol 2004; 286:G214-G224.
15. Miller M S, Galligan J J, Burks T F. Accurate measurement of intestinal transit in the rat. J Pharmacol Methods 1981; 6:211-217.
16. Turler A, Kalff J C, Moore B A, Hoffman R A, Billiar T R, Simmons R L, Bauer A J. Leukocyte-derived inducible nitric oxide synthase mediates murine postoperative ileus. Annals of Surgery 2006; 244:220-229.
17. Eskandari M K, Kalff J C, Billiar T R, Lee K K W, Bauer A J. LPS-induced muscularis macrophage nitric oxide suppresses rat jejunal circular muscle activity. American Journal of Physiology—Gastrointestinal & Liver Physiology 1999; 277:G478-G486.
18. Xiong H, Zhu C, Li F, Hegazi R, He K, Babyatsky M, Bauer A J, Plevy S E. Inhibition of interleukin-12 p40 transcription and NF-kappaB activation by nitric oxide in murine macrophages and dendritic cells. Journal of Biological Chemistry 2004; 279:10776-10783.

### Example 5

This example demonstrates that pretreatment with glycine prevents postsurgical ileus via immunomodulatory effects in the intestinal muscularis.

Methods: ACI-rats (180-220 g) underwent standardized surgical manipulation of the intestine. Glycine (170 mg) was injected intravenously 1 hour prior to surgery. Age matched animals treated with vehicle or valine (osmotic control) at appropriate time points and at equal volumes served as controls. Glycine receptors within the muscularis were investigated by immunohistochemistry. Postoperative mediator mRNA expression in the intestinal muscularis was determined by real-time RT PCR. Leukocyte extravasation was investigated in muscularis whole-mounts. Nitrite production was measured in muscularis cultures. In vivo transit of FITC-dextran was measured using geometric center analysis (GC). In vitro circular muscle contractility was assessed in a standard organ bath. Statistical analysis: unpaired Student t test, p<0.05, mean±SEM.

Results: Glycine receptors were immunohistochemically localized in resident muscularis macrophages and infiltrating leukocytes. Preoperative glycine injection significantly attenuated postoperative leukocyte recruitment by 27%. Intestinal manipulation caused a significant induction of IL-6 (8521 fold), TNF-α, (3.4 fold), ICAM-1 (21 fold), and iNOS (197 fold) at 3 hours postoperatively compared to non-operated vehicle treated controls. Glycine pretreatment alleviated the postoperative induction of inflammatory mediators significantly (IL-6: 2742 fold, TNF-α: 1.9 fold, ICAM-1: 14 fold, iNOS: 17 fold). Nitrite production increased significantly after intestinal manipulation. Glycine attenuated this response by 39%. Accordingly, glycine pretreatment ameliorated postoperative muscularis contractility (by 54%) and intestinal transit significantly.

Conclusion: The data indicate that preoperative glycine reduces postoperative ileus via the attenuation of muscularis inflammation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. In a method of performing a surgical procedure on a human patient, the improvement comprising administering to the patient interleukin-10 (IL-10), glycine, a COX-2 inhibitor, and a mast cell stabilizer in an amount and at a location sufficient to therapeutically or prophylactically suppress postoperative ileus in the human patient.

2. The method of claim 1, wherein the IL-10, glycine, COX-2 inhibitor, and mast cell stabilizer are administered to the patient prior to the surgical procedure.

3. The method of claim 1, wherein the IL-10, glycine, COX-2 inhibitor, and mast cell stabilizer are administered to the patient prior to completion of the surgical procedure.

4. The method of claim 1, wherein the IL-10, glycine, COX-2 inhibitor, and mast cell stabilizer are administered to the patient prior to completion of healing of a wound resulting from the surgical procedure.

5. The method of claim 1, wherein the IL-10, glycine, COX-2 inhibitor, and mast cell stabilizer are administered to the patient substantially concurrently.

6. The method of claim 1, wherein the IL-10, glycine, COX-2 inhibitor, and mast cell stabilizer are administered to the patient in a single pharmaceutical formulation.

7. The method of claim 1, wherein the COX-2 inhibitor is, celecoxib, meloxicam, rofecoxib, or valdecoxib or a pharmaceutically-acceptable salt or hydrate thereof.

8. The method of claim 1, wherein the mast cell stabilizer is azelastine, cromolyn, nedocromil, ketotifen, lodoxamide, nedocromil, olopatadine, or pemirolast or a pharmaceutically-acceptable salt or hydrate thereof.

9. The method of claim 1, wherein the suppression of postoperative ileus is prophylactic.

10. A method of suppressing postoperative ileus in a human patient undergoing a surgical procedure, the method comprising (a) conducting a surgical procedure on the human patient and (b) administering to the patient IL-10, glycine, a COX-2 inhibitor, and a mast cell stabilizer in an amount and at a location sufficient to therapeutically or prophylactically suppress postoperative ileus in the patient.

11. The method of claim 10, wherein the IL-10, glycine, COX-2 inhibitor, and mast cell stabilizer are administered to the patient prior to the surgical procedure.

12. The method of claim 10, wherein the IL-10, glycine, COX-2 inhibitor, and mast cell stabilizer are administered to the patient prior to completion of the surgical procedure.

13. The method of claim 10, wherein the IL-10, glycine, COX-2 inhibitor, and mast cell stabilizer are administered to the patient prior to completion of healing of a wound resulting from the surgical procedure.

14. The method of claim 10, wherein the IL-10, glycine, COX-2 inhibitor, and mast cell stabilizer are administered to the patient substantially concurrently.

15. The method of claim 10, wherein the IL-10, glycine, COX-2 inhibitor, and mast cell stabilizer are administered to the patient in a single pharmaceutical formulation.

16. The method of claim 10, wherein the COX-2 inhibitor is, celecoxib, meloxicam, rofecoxib, or valdecoxib or a pharmaceutically-acceptable salt or hydrate thereof.

17. The method of claim 10, wherein the mast cell stabilizer is azelastine, cromolyn, nedocromil, ketotifen, lodoxamide, nedocromil, olopatadine, or pemirolast or a pharmaceutically-acceptable salt or hydrate thereof.

18. The method of claim 10, wherein the suppression of postoperative ileus is prophylactic.

19. A pharmaceutical composition comprising, as active agents, IL-10, glycine, a COX-2 inhibitor, a mast cell stabilizer and a pharmaceutically-acceptable carrier.

20. The pharmaceutical composition of claim 19, which is formulated for local administration to the bowel of a patient.

21. The pharmaceutical composition of claim 19, which is formulated as a liquid, spray, foam, ointment or gel.


SEQUENCE LISTING (22 sequences)

SEQ ID NO: 1 - DNA, 21 bp, Artificial Sequence
  primer
atggcacagtcaaggctgaga

SEQ ID NO: 2 - DNA, 20 bp, Artificial Sequence
  primer
cgctcctggaagatggtgat

SEQ ID NO: 3 - DNA, 20 bp, Artificial Sequence
  primer
ctctgcgatgctcttccgag

SEQ ID NO: 4 - DNA, 20 bp, Artificial Sequence
  primer
aaggatttgctgcatggctg

SEQ ID NO: 5 - DNA, 20 bp, Artificial Sequence
  primer
cgtggcgtccatttacacct

SEQ ID NO: 6 - DNA, 19 bp, Artificial Sequence
  primer
ttagggcctcctcctgagc

SEQ ID NO: 7 - DNA, 21 bp, Artificial Sequence
  primer
gcccttcaggaacagctatga

SEQ ID NO: 8 - DNA, 23 bp, Artificial Sequence
  primer
tgtcaacaacatcagtcccaaga

SEQ ID NO: 9 - DNA, 22 bp, Artificial Sequence
  primer
ggagagatttttcacgacaccc

SEQ ID NO: 10 - DNA, 22 bp, Artificial Sequence
  primer
ccatgcataatttggacttgca

SEQ ID NO: 11 - DNA, 21 bp, Artificial Sequence
  primer
ggtgatcggtcccaacaagga

SEQ ID NO: 12 - DNA, 19 bp, Artificial Sequence
  primer
cacgctggctcagccactc

SEQ ID NO: 13 - DNA, 22 bp, Artificial Sequence
  primer
tcaattccagaaaccgctatga

SEQ ID NO: 14 - DNA, 20 bp, Artificial Sequence
  primer
caccagcatcagtcccaaga

SEQ ID NO: 15 - DNA, 19 bp, Artificial Sequence
  primer
caggtcgctcagggtcaca

SEQ ID NO: 16 - DNA, 21 bp, Artificial Sequence
  primer
cagaggcaaggaggaaacaca

SEQ ID NO: 17 - DNA, 24 bp, Artificial Sequence
  primer
ccaactctcactgaagccagctct

SEQ ID NO: 18 - DNA, 19 bp, Artificial Sequence
  primer
caggcccagaagcatgaca

SEQ ID NO: 19 - DNA, 21 bp, Artificial Sequence
  primer
ctcactggcaggaaatcatcc

SEQ ID NO: 20 - DNA, 21 bp, Artificial Sequence
  primer
acctcgtggagacgctttaca

SEQ ID NO: 21 - DNA, 26 bp, Artificial Sequence
  primer
tgaaggtcggtgtgaacggatttggc

SEQ ID NO: 22 - DNA, 24 bp, Artificial Sequence
  primer
catgtaggccatgaggtccaccac